(19) United States
(12) Patent Application Publication
(10) Pub. No.: US 2003/0211457 A1
(43) Pub. Date: Nov. 13, 2003

(54) Methods and kits for testing mutagenicity

(75) Inventors: Luman Wing, San Diego, CA (US); Michael Saghbini, San Diego, CA (US)

Correspondence Address: KEITH JOHNSON, ESQ., TRANSGENOMIC, INC., 12325 EMMETT STREET, OMAHA, NE, 68164, US

(73) Assignee: Transgenomic, Inc., San Jose, CA

(21) Appl. No.: 10/273,601

(22) Filed: Oct. 17, 2002

Related U.S. Application Data
(60) Provisional application No. 60/371,039, filed on Apr. 8, 2002.
(60) Provisional application No. 60/380,359, filed on May 13, 2002.

(51) Int. Cl. C12Q 1/00; C12Q 1/68; C12N 1/20

(52) U.S. Cl. 435/4; 435/6; 435/252.3

(57) ABSTRACT

In one aspect, methods and kits for determining the mutagenic potential of a test substance. The method includes exposing a tester strain (such as Salmonella typhimurium) to the substance, wherein the tester strain includes a gene (such as the histidine gene) having a preexisting mutation conferring auxotrophy, and the mutation is located at a pre-determined position in the gene, growing the tester strain in growth media lacking histidine, and detecting the presence of a back-mutation at the position by analysis of the nucleic acid. The tester strain can be selected from TA98, TA100, TA102 TA1535, TA1537, TA1538, and TA97. The test substance can be any of a wide variety of compounds such as petroleum extracts, pesticides, cosmetics, adhesives, herbicides, hair dyes, and pharmaceuticals. The detecting step can include one or more conventional mutation detection methods. Also provided are kits for conducting the method. The kits can include one or more tester strains, PCR primers, positive control compounds, and DNA polymerase.

### CROSS-REFERENCE TO RELATED APPLICATIONS

This application is a non-provisional U.S. patent application under 35 U.S.C. §111 (a) and claims priority from the following co-pending, commonly assigned provisional applications, each filed under 35 U.S.C. §111(b): Ser. No. 60/371,039 filed Apr. 8, 2002 and Ser. No. 60/380,359 filed May 13, 2002.

### FIELD OF THE INVENTION

The invention is in the fields of biochemistry and toxicology.

### BACKGROUND OF THE INVENTION

The traditional Ames test is an FDA approved bacterial mutation assay designed to identify substances that can produce genetic damage. This assay is valuable because of the high correlation between mutagenic response and rodent carcinogenicity. The Ames assay uses a number of modified Salmonella strains with preexisting mutations in various regions of the histidine operon that render the cells unable to grow in the absence of histidine (auxotrophy). Upon exposure to test substances in the presence or absence of an exogenous mammalian metabolic activation system (liver S9 fraction) the genetic deficiency is corrected restoring histidine-independence (prototrophy). Correction can occur at the site of the preexisting mutation (hot spot) or nearby. Different strains are active with different classes of compounds. Table 1 outlines the reversion events detected by the various Ames strains.

TABLE 1

| Mutation | | | LPS | |
|---|---|---|---|---|
| (strain) | DNA target | Reversions detected | defect | Plasmid |
| hisG46 | GGG | Base-pair substitution | rfa | No |
| TA1535 | | | | plasmid |
| TA100 | | | | |
| hisD3052 | CGCGCGCG | Frameshifts | rfa | No |
| TA98 | | | | plasmid |
| hisC3076 | +1 frameshift | Frameshifts | rfa | No |
| TA1537 | (near CCC run) | | | plasmid |
| hisD6610 | CCCCCC | Frameshifts | rfa | |
| TA97 | (+1 cytosine at | | | |
| | run of C's) | | | |
| hisG428 | TAA (ochre) | Transitions/transversions | rfa | pAQ1 |
| TA102 | | | | |

The standard Ames protocol involves exposing 100 ul of an overnight culture to 50 ul of test substance dissolved in either water or DMSO in the presence of 500 ul of metabolic activation system (rat or hamster liver S9 fraction) or a phosphate buffer (no S9). Following an optional 20-30 minute incubation at 37° C. the exposed cells are mixed with 2 ml of top agar at 40-50° C. containing limited histidine and plated on minimal plates. After 48 hours incubation at 37° C. the number of colonies are recorded. Each compound is tested in 5 doses covering a range of at least three logs with each dose tested in triplicate. A greater than 2-fold increase in reversion rate above background in any strain classifies the compound tested as mutagenic. Table 2 lists the number of background revertant colonies observed in a standard Ames assay.

TABLE 2

| Strain | +S9 | −S9 |
|---|---|---|
| TA97 | 75-200 | 100-200 |
| TA98 | 20-50 | 20-50 |
| TA100 | 75-200 | 75-200 |
| TA102 | 100-300 | 200-400 |
| TA1535 | 5-20 | 5-20 |
| TA1537 | 5-20 | 5-20 |

The traditional Ames test is used world-wide as an initial screen to detect mutagenic potential of new compounds. A battery approach is used involving different strains to test for Ames mutagenicity. Testing is first done in strains TA98 and TA100 with and without 10 and 30% S9. If positive results are obtained, the test is repeated under the same conditions that gave the positive result to confirm it and classify the substance as mutagenic. If the substance is not mutagenic or weakly mutagenic, it is tested in strains TA97 and TA1535 with and without the S9 fraction and repeated to confirm a positive result. A weak response in strain TA97 may be repeated in strain TA1537. Strains TA102 and TA104 may be used if it is suspected that the chemical may induce oxidative damage or be a DNA cross-linking agent.

The traditional Ames assay is lengthy, taking about 3-5 day to complete, requires the use of multiple culture plates, requires milligram quantities of compound, is difficult to adapt to high throughput screening procedures, has a limited sensitivity, and is expensive.

There is a need for quicker, more convenient assays that are less costly, utilize minimal quantities of test compound, have greater sensitivity, and can be used in automated high throughput screening technologies.

### SUMMARY OF THE INVENTION

In one aspect, the invention provides a method for screening a test substance suspected of being a mutagen. In one embodiment, the method comprises a) providing a tester strain of Salmonella typhimurium, wherein the tester strain comprises a histidine gene having a preexisting mutation conferring auxotrophy, the mutation located at a pre-determined position in the gene, b) determining a first level of back-mutation in the nucleic acid sequence of the gene in tester strain which have not been exposed to the substance, c) exposing tester strain to the substance and determining a second level of back-mutation in the nucleic acid sequence of the gene, d) comparing the first level with the second level. The method can used to determine whether the substance is mutagenic. The preexisting mutation can be a base substitution or a frame shift mutation. The method can include, amplifying, such as by PCR, a pre-selected region of the histidine gene, wherein the position is within the region. The determination of a back mutation in step (b) and step (c) can be performed by a method selected from the group consisting of direct sequencing, minisequencing, pyrosequencing, single-stranded conformation polymorphism, denaturing gradient gel electrophoresis, chemical cleavage, cleavage by mismatch endonuclease, allele specific oligonucleotides, ligase mediated detection of mutations, invader assay, and denaturing high performance liquid chromatography. Examples of test substance include petroleum extract, pesticides, cosmetics, adhesives, food coloring, herbicides, hair dyes, and pharmaceuticals. The tester strain can be selected from the group consisting of TA98, TA100, TA102, TA104, TA1535, TA1537, TA1538, and TA97. The exposing step (c) can include S9 homogenate. The method can include correlating the level of DNA having the back-mutation with the concentration of the substance in the media. Step (d) can include determining whether the test substance is a mutagen. The test compound can be a hydrocarbon mixture (e.g. or petroleum origin), and wherein the method includes extracting the hydrocarbon mixture with a solvent effective for removing mutagenic compounds from the mixture. The method can include the use of an optimal amount of induced liver homogenate is included as a metabolic activator in the exposing in step (c). The exposure can include S9 liver homogenate, such as Aroclor 1254-induced rat S9, Aroclor 1254-induced hamster S9, Aroclor 1254-induced rat S9, or Aroclor 1254-induced hamster S9. Example of suitable solvent for extraction include DMSO, 1-methyl-2-pyrrolidinone and N,N-dimethylformamide.

In another aspect, the invention concerns a method for determining the mutagenic potential of a putative mutagen. An embodiment includes a) exposing a tester strain of Salmonella typhimurium to the mutagen, wherein the tester strain comprises a histidine gene having a preexisting mutation conferring auxotrophy, the mutation located at a predetermined position in the gene, b) growing the tester strain in growth media lacking histidine, c) detecting the presence of a back-mutation at the position, wherein the presence of the back-mutation is correlated with the mutagenic potential of the mutagen. The putative mutagen can be a physical treatment such as exposure to ultraviolet light or other radiant source.

In another aspect, the invention provides a method for evaluating the potential mutagenicity of a test compound. The method includes a) subjecting an inoculum of a histidine deficient mutant strain of Salmonella typhimurium to incubation in the presence of the compound wherein the strain comprises DNA possessing a preexisting mutation at a pre-determined position in the histidine biosynthetic operon, b) determining the presence in the incubation of DNA having a back-mutation at the position, wherein the DNA having a back-mutation is indicative of the mutagenicity of the compound. The test compound can be a hydrocarbon mixture, and the method can include extracting the hydrocarbon mixture with a solvent effective for removing mutagenic compounds from the mixture. Preferably an optimal amount of induced liver homogenate is included as a metabolic activator in the incubation.

In yet another aspect, the invention provides a method for determining the mutagenic potential of a petroleum extract. The method includes a) exposing a tester strain of Salmonella typhimurium to the extract, wherein the tester strain comprises a histidine gene having a preexisting mutation conferring auxotrophy, the mutation located at a predetermined position in the nucleic acid sequence of the gene, b) growing the tester strain in growth media lacking histidine, c) detecting the presence of a back-mutation in the nucleic acid sequence at the position, wherein the extract is classified as a mutagen if the level of back-mutation is above a pre-determined background level. An example of such a predetermined level is twice the background level.

In still another aspect, there is provided a method for determining the mutagenic potential of a test substance. The method comprises a) a step for exposing a tester strain of Salmonella typhimurium to the substance, wherein the tester strain comprises a histidine gene having a preexisting mutation conferring auxotrophy, the mutation located at a pre-determined position in the gene, b) a step for growing the tester strain in growth media lacking histidine, and c) a step for detecting the presence of a back-mutation at the position, wherein the presence of the back-mutation correlates with the mutagenic potential of the substance.

In a further aspect, there is provided a method for screening a test substance suspected of being a mutagen. The method comprises a) a step for providing a tester strain of Salmonella typhimurium, wherein the tester strain comprises a histidine gene having a preexisting mutation conferring auxotrophy, the mutation located at a pre-determined position in the gene, b) a step for determining a first level of back-mutation in the nucleic acid sequence of the gene in tester strain which have not been exposed to the substance, c) a step for exposing tester strain to the substance and determining a second level of back-mutation in the nucleic acid sequence of the gene, d) a step for comparing the first level with the second level.

In an additional aspect, the invention provides a kit or kits for evaluating the mutagenicity of a test compound. A kit can include one or more of the following: a tester strain of Salmonella typhimurium, wherein the tester strain is selected from the group consisting of TA98, TA100, TA102, TA104, TA1535, TA1537, TA1538, and TA97; pre-selected PCR primers specific for amplification of a region of the Salmonella histidine operon in the strain, wherein the region contains a preexisting mutation that confers auxotrophy in the strain, wherein the primers generate an amplicon having a length of about 150 base pairs to about 500 base pairs; reference DNA fragment corresponding to the region for use in a hybridization protocol with the amplicon; a plurality of tester strains of Salmonella typhimurium, wherein said tester strains are selected from two or more of the group consisting of TA98, TA100, TA102, TA104, TA1535, TA1537, TA1538, and TA97, wherein the tester strains are preferably provided in a ready-to-use format; a standard mixture of a tester strain and cells including the back mutation; S9 fraction in a separate container; one or more positive control compounds, wherein the control compound can include HC235; a proofreading DNA polymerase such as Pho polymerase or Taq polymerase; a pre-selected PCR primers specific for amplification of a region of the Salmonella histidine operon in the strain, wherein the region contains a preexisting mutation that confers auxotrophy in the strain, wherein the primers generate an amplicon having a length of about 150 base pairs to about 500 base pairs; a reference DNA fragment corresponding to the region for use in a hybridization protocol in DHPLC.

In a further aspect the invention concerns a method for determining the mutagenic potential of a test substance, the method including a) exposing a bacterial tester strain to said substance in a first media, wherein said tester strain comprises a gene having a preexisting mutation conferring auxotrophy, said mutation located at a pre-determined position in said gene, b) growing said tester strain in selective enrichment media to enrich for back-mutation revertants, and c) detecting the presence of a back-mutation, at the nucleic acid level at said position or nearby, wherein the presence of said back-mutation is distinguished from spontaneous revertant mutations and correlates with the mutagenic potential of said substance. Steps (a) and step (b) can be combined into a single step. The tester strain can include Salmonella and the gene can be the histidine biosynthetic gene. The tester bacteria can include E. coli and the gene can be the tryptophan biosynthetic gene. In one embodiment of the method. In one embodiment, the first media and the selective enrichment media are devoid of histidine. In another embodiment, the first media and said selective enrichment media contain a limiting amount of histidine. In another embodiment, the first media and the selective enrichment media contain a limiting amount of Luria Broth. In yet another embodiment, the first media and said selective enrichment media contain a limiting amount of tester strain growth media to support limited growth of said tester strain while allowing selective enrichment of said back-mutation revertants. Preferably, the enrichment of said back-mutation revertants occurs to a level sufficient for detection of said back mutation at the nucleic acid level. The detection can include a method selected from the group consisting of direct sequencing, minisequencing, pyrosequencing, single-stranded conformation polymorphism, denaturing gradient gel electrophoresis, chemical cleavage, cleavage by mismatch recognition endonuclease, allele specific oligonucleotides, ligase mediated detection of mutations, invader assay, sizing, and denaturing high performance liquid chromatography. In the method, a plurality of tester strains can be used in step (a). The method is preferably repeated at different doses of test substance. The tester strain can be used in limited amounts to control the level of said background revertants. The tester strain is preferably exposed multiple independent times to said test substance to account for said background revertants. The multiple independent times preferably represent a statistically significant number of repeats to establish a 2-fold increase over said background revertants. The tester strain can comprise a plurality of tester strains, in any combination, selected from two or more of the group consisting of TA98, TA100, TA102, TA1535, TA1537, TA1538, and TA97. The tester strain can include a limited amount of said plurality of tester strains to control the level of said background revertants. The back-mutation can be an insertion or deletion. The test substance is preferably dissolved in liquid solvent selected the group consisting of DMSO, glycerol formal, dimethyl formamide, formamide, acetonitrile, 95% ethanol, acetone, ethyl glycol, dimethyl ether, 1-methyl-2-pyrrolidone, tetrahydrofurfuryl alcohol, water, and tetrahydrofuran. The solvent is preferably used at a concentration that is not lethal to tester strain.

### BRIEF DESCRIPTION OF THE DRAWINGS

FIG. 1 shows a chromatographic elution profile obtained from DHPLC analysis of DNA from Salmonella cells not exposed to sodium azide.

FIG. 2 shows a first chromatographic elution profile obtained from DHPLC analysis of DNA from Salmonella cells exposed to sodium azide.

FIG. 3 shows a second chromatographic elution profile obtained from DHPLC analysis of DNA from Salmonella cells exposed to sodium azide.

FIG. 4 shows a third chromatographic elution profile obtained from DHPLC analysis of DNA from Salmonella cells exposed to sodium azide.

FIG. 5 shows a fourth chromatographic elution profile obtained from DHPLC analysis of DNA from Salmonella cells exposed to sodium azide.

FIG. 6 shows a fifth chromatographic elution profile obtained from DHPLC analysis of DNA from Salmonella cells exposed to sodium azide.

FIG. 7 shows a chromatographic elution profile obtained from DHPLC analysis of DNA from Salmonella cells not exposed to UV light.

FIG. 8 shows a chromatographic elution profile obtained from DHPLC analysis of DNA from Salmonella cells exposed to UV light. The exposure time was 3 sec.

FIG. 9 shows a chromatographic elution profile obtained from DHPLC analysis of DNA from Salmonella cells exposed to UV light. The exposure time was 15 sec.

FIG. 10 shows a chromatographic elution profile obtained from DHPLC analysis of DNA from Salmonella cells exposed to UV light. The exposure time was 30 sec.

FIG. 11 illustrates growth of Salmonella colonies on agar plates.

### DETAILED DESCRIPTION OF THE INVENTION

In a general aspect, Applicants have invented novel methods and kits for determining the mutagenic potential of test substances or treatments.

The traditional AMES test is a bacterial reverse mutation assay specifically designed to detect a wide range of chemical substances that can produce genetic damage that leads to gene mutation. The AMES test employs several histidine dependent Salmonella strains each carrying different preexisting mutations in various genes in the histidine operon. These preexisting mutations act as hot spots for mutagens that cause DNA damage via different mechanisms.

In a general aspect, the present invention provides a method in which a tester strain of bacteria is exposed to a test substance. A back-mutation in a preexisting mutation in a biosynthetic gene is detected as described herein in order to assess the mutagenic potential of the substance. Test substances amenable to the method described herein include a diverse group of compounds, such as pesticides, cosmetics, adhesives, herbicides, hair dyes, food additives, preservatives, and pharmaceuticals. Other examples of test compounds include ingredients found in complex hydrocarbon mixtures encountered in petroleum refinery streams, and preferably a dimethylsulfoxide (DMSO) extract of such mixtures (as described in U.S. Pat. No. 4,499,187). Although DMSO is the presently preferred solvent for extracting such a hydrocarbon mixture, it is contemplated that other solvents such as 1-methyl-2-pyrrolidinone and N,N-dimethyl formamide can be used.

For example, a complex hydrocarbon mixture such as a petroleum refinery stream or product may be evaluated for possible carcinogenic activity by extracting the hydrocarbon mixture with a suitable solvent, such as dimethylsulfoxide (DMSO), and subjecting an inoculum of a histidine deficient mutant strain of Salmonella typhimurium to incubation in the presence of an aliquot of the extract together with, as metabolic activator, an optimal amount of liver homogenate. Examples of analysis of petroleum compounds are described in the Examples hereinbelow.

Many complex mixtures of petroleum hydrocarbons that demonstrate dermal carcinogenicity in rodents are undetected or only induce marginal responses in the standard bacterial mutagenicity assay. Blackburn et al. (Cell Biol. Toxicol. 1:67 (1984); U.S. Pat. No. 4,499,187) described a modified S. typhimurium assay with improved sensitivity to complex mixtures of petroleum hydrocarbons with boiling points≧500° F. The Blackburn method overcame two difficulties in testing petroleum hydrocarbons. First, by extracting the oil sample with dimethyl sulfoxide, aqueous compatible solutions were obtained. Second, the metabolic activation system was enhanced by using an 80% hamster liver S9 mix with a 2×NADP concentration rather than the standard 10% rat liver S9. Blackburn et al. (Cell Biol. Toxicol. 2:63 (1986)) further reported that when using tester strain TA98 in the modified assay, there was excellent correlation between the slope of the initial portion of the mutagenicity curve (termed the mutagenicity index) and the carcinogenic potency of the oil.

Examples of known mutagens include nitrosamines, polycyclic hydrocarbons, fungal toxins, aromatic amines, nitrofuran carcinogens, various antineopleastic agents, antibiotic carcinogens such as adriamycin, daunomycin, and mitomycin C, β-naphthylamine, benzidine, cigarette smoke condensates, bis-choromethyleterh, 4-aminobipheny, azoxymethane, aflatoxin B1, sterigmatocystin, furylfuramide, nitrofuran carcinogens, acetylenic diarylcarbamates, benzo[a]pyrene, 2-acetylaminofluorene, 2-aminofluorene, nitroquinolline-N-oxide, ethylene oxide, hydrazine sulfate, bleomycin, tert-butyhydroperoxide, HC235 extract, methyl methanesulfonic acid, ICR191, 9-amino acrydine, Danthron, cyclophosphamide, ethyl methanesulfonate, and sodium azide. A list of additional chemicals evaluated as mutagenic is described in Prival et al. (Mutation Research 412:251-260 (1998)). Such known mutagens are used as positive control compounds in assays for mutagenicity as described herein.

Another aspect of the present invention concerns the use of tester strains of bacteria. Examples of preferred tester strains include nutritional mutants of Salmonella typhimurium and E. coli that are used in the art of mutagen testing, such as described by Ames, et al. Mutation Res. 31:347-364 (1975); Maron et al, Mutation Research 113:173-215 (1983); and by Putman, et al. (Toxicology Testing Handbook, Jacobson-Kram and Keller eds., Marcel Dekker New York, (2000) pp.127-194). These strains are widely used in conventional reversion tests. The Salmonella and E. coli strains that can be used in the instant invention each have a defect (i.e. alteration) in one of the genes involved in histidine and tryptophan biosynthesis, respectively. In the case of Salmonella, the defect renders the cell dependent on exogenous histidine. Unless the cell experiences a mutation that reverses the dysfunctional gene back to the wild type (i.e. a back-mutation), the cell becomes disabled when the exogenous histidine is exhausted. For this reason, this assay is referred to as a reverse or back mutation assay. For the purposes of clarity, and not by way of limitation, the use of Salmonella strains will be described hereinbelow.

In the present invention, preferred Salmonella tester strains include TA100, TA1535, TA98, TA1538, TA1537, TA97, TA104, and TA102. Each of these strains carry different mutations in various genes in the histidine operon. Tester strain genotypes are shown in Table 1 (Putman et al. (2000)). These strains complement each other by detecting base-pair or frameshift mutations. Batteries of strains can be used, are preferably selected in accordance with regulatory requirements, and can also be selected with regard to the nature of the chemical. For example, if the specific properties of a chemical make one strain particularly sensitive, that strain should most likely be included in the battery. All strains are histidine dependent by virtue of an engineered mutation in the histidine operon. Additional mutations/genetic alterations that have made the tester strains more sensitive to chemical mutagens are listed below (as reviewed in Mortelmans et al (2000)):

A deletion mutation through the uvrB-bio genes in all strains, except TA102. The uvr B deletion mutation eliminates the accurate excision repair mechanism, thereby allowing more DNA lesions to be repaired by the error-prone DNA repair mechanism. The deletion through the biotin gene makes the bacteria biotin dependent.

A mutation (rfa) in all strains that lead to a defective lipolysaccharide (LPS) layer that coats the bacterial surface, making the bacteria more permeable to bulky chemicals.

Introduction of plasmid pKM101 in strains TA1535 and TA1538 resulting in the corresponding isogenic strains TA100 and TA98 and in strains TA97 and TA102. Plasmid pKM101 enhances chemical and UV-induced mutagenesis via an increase in the error-prone recombinational DNA repair pathway. The plasmid confers ampicillin resistance, which is a convenient marker to detect the presence of the plasmid.

Insertion of the mutation hisG428 on the multi-copy plasmid pAQ1 which was introduced in strain TA102 with the aim of amplifying the number of target sites. To enhance the ability of this strain to detect DNA cross-linking agents, the uvrB gene was retained making the bacterium DNA repair proficient.

The complete sequence of the histidine operon for Salmonella typhimurium is available (NCBI Accession No. J01804). As described herein, Applicants have used this sequence to design PCR primers to amplify various regions of the operon. In particular, and as shown in the Examples herein, regions that include preexisting mutations were amplified in various strains and subjected to DNA sequencing and other analyses both before and after exposure of the strains to mutagens. Applicants have discovered that back-mutation events could be detected by these direct genetic analyses and were associated with reversion to prototrophy. For example, Applicants have verified, using conventional DNA sequencing, that strain TA102 contains a T at position 1521 in the hisG428 gene, and have verified that revertants arising after exposure to mutagen contain a C at this position. TA102 has a hisG428 gene mutation located on a multicopy plasmid, pAQ1. This strain is particularly sensitive to the activity of oxidative mutagens and cross-linking agents (Levin et. al., PNAS 79:7445-9, 1982). In another example, Applicants have verified that strains TA100 and TA1535 contain a C at position 1101 in hisG46, and have shown that revertants after exposure to mutagen contain a T at this position. Applicants have also verified that strain TA98 contains a C deleted form a stretch of CCC starting at position 2798, and have verified by sequencing that reversion with the mutagen daunomycin causes a CG deletion from a stretch of 4 CG starting at position 2783. This reversion event represents the major class of revertants observed in TA98. Other possible revertants include, but may not be limited to, the addition of C residue about 6-7 bases downstream of the engineered mutation (i.e., the preexisting mutation at 2798) and a 10 nucleotide insertion about 28 bases upstream from the engineered site (Isono et al., PNAS 71, 5, 1612-1617). Thus, for TA98, the back-mutation can involve alterations at positions that are within the vicinity (i.e. within about 30 base pairs) of the position of the preexisting mutation.

Another aspect of the present invention concerns conditions and methods for exposing a tester strain to a test substance and for providing conditions suitable for the growth of revertant cells. In a general embodiment, there is provided a method in which a tester strain is subjected to a first stage incubation in which the strain is exposed to the test substance, or test treatment, in a first liquid media, followed by a second stage incubation in which the strain is diluted into liquid selective enrichment growth media. In one preferred embodiment, and prior to the first stage incubation, an overnight culture of a selected Salmonella strain is prepared in 10 ml liquid media. A preferred media is Luria Broth, which can contain antionbiotic, such as ampicillin (e.g. at 50 ug/ml) or tetracycline (e.g. at 10 ug/ml). Cells are preferably incubated for 18 h at 37° C., with rotation at 250 rpm to a level of about 1×10⁹ cells/ml. The tester strain can be supplied form an overnight culture grown in liquid media or on plates, fresh or few weeks old. It can also come from a frozen stock or lyophilized cells.

In the first stage incubation, an aliquot of the tester strain from the overnight culture is incubated in a first liquid media. The aliquot of tester strain can be washed and resuspended in histidine-deficient media. The aliquot can be 0.05 ul to 75 ul, preferably 0.1 ul to 10 ul, and optimally 1 ul. The final volume of the first stage incubation can be in the range of 0.01 ml to 1 ml, preferably 0.05 ml to 0.5 ml, and optimally 0.1 ml. The first stage incubation is preferably carried out at 37° C., and for a period in the range of 0 minutes to about 5 hours, preferably for about 2 hours. This incubation includes the test compound (i.e. test article) at a dose ranging from 0.1 ug to 1 mg. Positive controls (i.e. with a known mutagen) and vehicle controls (i.e. lacking test compound) are preferably included in order to validate the method. The test compound can be dissolved in a solvent, and preferably a solvent that does not interfere with tester strain growth. Examples of suitable solvent include DMSO, glycerol, glycerol formal, dimethyl formamide, formamide, acetonitrile, 95% ethanol, acetone, ethyl glycol, dimethyl ether, 1-methyl-2-pyrrolidone, tetrahydrofurfuryl alcohol and tetrahydrofuran.

The amount of overnight culture used in the first stage incubation can be selected in order to adjust and control the amount of background spontaneous revertants. For example, using tester strain TA1535, it was found that 1 ul of overnight culture was sufficient to reduce spontaneous revertants to a non-existent level of detection (as detected using the nucleic acid analytical methods as described hereinbelow). Since each tester strain reverts at a different frequency, optimization of starting cells can be used to control the background level. If the background cannot be reduced to zero, a statistically significant number of repeats per dose of test substance is preferably performed to obtain at least a 2-fold increase over this background.

The first stage incubation in the first liquid media can include S9 microsomal fraction for those mutagens that require activation to their active forms (i.e. promutagens). The S9 is preferably from mammalian liver. Examples of suitable S9 include microsomes obtained from mouse, rat or hamster liver, and are preferably from animals induced with agents such as PCB or Aroclor 1254. S9 can be prepared by conventional methods or obtained commercially (Maron et al. (1983)). The S9 is used with an NADPH activating system. The amount of S9 can be optimized using standard test compounds to give results that are reproducible. The optimal amount can be determined from a series of tests made with increasing amounts of a test compound. The optimal amount of S9 can also be determined from a series of tests made a differing levels of S9.

In the second stage incubation, the cells from the first stage are diluted by addition of selective enrichment media that lacks histidine. This media promotes or enhances the growth of mutant (i.e. revertant) Salmonella. The volume of the second stage incubation can be 0.02 ml to about 10 ml, preferably about 0.5 ml to about 1 ml, and optimally about 0.8 ml. In one preferred embodiment, the selective growth media is added to a final volume of 0.8 ml and consists of M9 media containing 2% glucose, minimal salts, biotin, and is incubated at 37° C. for 18 h, with rotation at 250 rpm. In other embodiments, the incubation step can range from 1 to 24 hours, preferably 10 to 20 hours, and more preferably 18 hours, an can include rotation. The incubation temperature can be in the range of about 25° C. to about 40° C., and is preferably 37° C. It is preferred to amplify the mutagen-induced revertant cells to a level such that they represent 1% to 99% of the total cell population, preferably 5% to 50%, and more preferably about 25% to about 50% of the total population. Preferably, the number of revertant cells is sufficient such that back-mutations can be detected using the selected methodology, and will depend on the method selected. For example, in DHPLC, the sensitivity is about 5-10%, while for sequencing, the sensitivity is about 20%.

In an alternative embodiment, only a single incubation is used, combining the first and second stages into a one-step process. In this procedure, the exposure media and selective enrichment media are combined into a single (e.g. 0.9 ml) incubation (combined first media and enrichment media) allowing simultaneous test substance exposure and revertant enrichment. This combined media can be added to a test substance from a master stock containing all necessary components (i.e. tester strain, S9 and media components). The incubation volume can be in the range of 0.01 ml to 10 ml, preferably about 0.5 ml to 1 ml and optimally about 0.9 ml. In this embodiment, the incubation temperature can range from 40° C. to 25° C., and is preferably 37° C. The incubation time range from about 1 hour to about 24 hours, more preferably about 6 to 20 hours and optimally about 18 hours.

Where appropriate, and if not already provided by the overnight culture, LB can be added to either stage of the two-stage process, and to the one-step process to a final concentration in the range of 0 to 5%, preferably 0 to 2% and optimally about 0.8%. This limited amount of LB allows limited tester strain growth while preferentially enriching for revertants. Other media and media components that can supply ingredients (i.e. histidine) required for limited growth of the tester strain can also be used. Applicants have found that the extent of LB addition is dependent on starting amount of tester strain and may not be required if sufficient amounts of tester strain cells are initially used such that the tester strain represents a range of 1-99% of the population, preferably, 5 to 50% and more preferably 25 to 50%.

The first media in the two-stage process and the combined media in the one stage process, containing the tester strain and all necessary components excluding the test compound in a any desired combination, can be preincubated for up to several hours at a temperature ranging form 25° C. to 60 ° C. for a duration of 0 to 6 hours, preferably around 2-3 hours, to better prepare the cells for a mutagenic response. Furthermore, cells in the presence of test compound can be incubated for a period of up to several hours, preferably around 30 minutes at temperatures ranging form 4° C. to 70° C., with several incubations at different temperatures in any order, prior to the enrichment incubation period, to better enhance the mutagenic response. Also a process that delivers electric shock (i.e. electroporation) or a chemical shock can be used for that purpose.

The enrichment media in the two-stage process and the combined media in the one-step process can be modified, to include an indicator compound, to monitor bacterial toxicity induced by test substance at different doses. Bromocresol purple, a pH indicator, is one such compound. It changes color form yellow to purple in response to the acidic environment generated by a growing culture.

Steps of the two-stage process and the one-step process can be carried out in multi-well vessels including but not limited to 24, 48, 96 and 384 wells plates, where appropriate solutions can be prepared as master stocks and properly aliquoted. Cells and other components can be provided in any combination, in a ready to use format, lyophilized or hydrated. After processing by either the two-stage process or the one-step process the cells are ready for mutation detection at the DNA level using a variety of methods. In some embodiments, it is preferred to premix a tester strain and a revertant population of cells. A premixed population can facilitate mutation detection by techniques that require a reference sample of DNA in order to detect such mutations, as described hereinbelow. The revertant cells in the mixed population provide a source of “endogenous” reference DNA.

Examples of methods for screening mutant sites within a gene that do not require a reference sample include: direct sequencing, minisequencing and pyrosequencing (U.S. Pat. No. 6,258,568). Examples of screening methods that preferably use mixed populations include single-stranded conformation polymorphism (SSCP), allele specific oligonucleotides (ASOs), ligase mediated detection of mutations, and the invader assay and sizing methods. Other screening methods that preferably use reference material (e.g., a mixed revertant-tester strain population) include: denaturing gradient gel electrophoresis (DGGE), cleavage using mismatch recognition endonucleases or chemicals, and denaturing high performance liquid chromatography (DHPLC). Nucleic acid sizing (e.g. by IP-RP-HPLC) does not require the use of reference DNA for a hybridization procedure, and may be especially useful if back-mutation involves addition or deletion of one or more bases.

DNA obtained from the second stage incubation, or the combined incubation, can be analyzed for the presence of a back-mutation using a variety of conventional methods. Examples of methods for screening for mutant sites within a gene include single-stranded conformation polymorphism (SSCP), denaturing gradient gel electrophoresis (DGGE), cleavage using mismatch recognition endonucleases or chemicals, allele specific oligonucleotides (ASOs), ligase mediated detection of mutations, minisequencing, pyrosequencing (U.S. Pat. No. 6,258,568), invader assay, and denaturing high performance liquid chromatography.

In single-stranded conformation polymorphism (SSCP), DNA is denatured and then immediately run on a non-denaturing gel. The secondary structures of wild-type strands (i.e. strands having the preexisting mutation) or back-mutant single strands differing by a single base are usually sufficiently different to result in different migration rates on polyacrylamide gels.

A reliable way to detect mutations is by hybridization of the putative mutant strand in a sample with the wild type strand (Lerman, et al., Meth. Enzymol. 155:482 (1987)). If a mutant strand is present, then, typically, two homoduplexes and two heteroduplexes will be formed as a result of the hybridization process. Hence separation of heteroduplexes from homoduplexes provides a direct method of confirming the presence or absence of mutant DNA segments in a sample.

In denaturing gradient gel electrophoresis (DGGE), either homoduplex or heteroduplex double stranded DNA is electrophoresed under denaturing conditions of increasing concentration until the last domain is denatured, and migration of the DNA through the gel is retarded. DNA sequences differing by a single base pair migrate at different rates along the gel, thereby allowing detection of a polymorphic site, if present.

Mismatch endonucleases or chemicals can cleave at a site of mismatch in heteroduplex molecules (see for example U.S. Pat. Nos. 6,391,557; 6,027,898; and 5,869,245). A preferred enzyme cleaves dsDNA hybrids wherever there is a mismatch. The chemical cleavage method is based upon a similar principle but uses chemicals such as organometallic DNA intercalators incorporating rhodium or ruthenium. Other chemicals include hydroxylamine and osmium tetroxide which can be used to distinguish between mismatched C or T nucleotides, respectively. The position of the mismatch (e.g., the mutation) is defined by sizing on gel electrophoresis or HPLC after cleavage at the reactive position by piperidine.

Allele-specific oligonucleotide probes (ASOs) are probes that are designed to hybridize selectively to either a normal or a mutant allele, where the probes are developed to distinguish between the normal and mutant sequence. This is done by altering the stringency of hybridization to a level at which each of the oligonucleotides will anneal stably only to the sequence to which it is perfectly complementary and not to the sequence with which it has the single mismatch. ASO if properly labeled can be used in conjunction with real time PCR in a Taqman like assay for immediate detection.

The ligase-mediated method for detecting mutations makes use of the fact that the ends of two single strands of DNA must be exactly aligned for DNA ligase to join them. In utilizing this technique, oligonucleotides complementary to the target sequence, 5′ to and including the mutation site, are synthesized and labeled. A third oligonucleotide complementary to the common sequence 3′ to the mutation site is synthesized and also labeled. The oligonucleotides are then hybridized to strands of the target. In cases in which the 5′ and 3′ oligonucleotides form a flush junction that can be joined by DNA ligase, ligation occurs. However, a single base pair mismatch occurring between the normal 5′ oligonucleotide and the mutation site is sufficient to prevent the ligase from acting and can readily be detected.

A common approach to analysis of DNA polymorphisms relies on variations in the lengths of DNA fragments produced by restriction enzyme digestion. The polymorphisms identified using this approach are typically referred to as restriction fragment length polymorphisms or RFLPs. Polymorphisms involving variable numbers of tandemly repeated DNA sequences between restriction enzyme sites, typically referred to as microsatellites or variable numbers of tandem repeats (VNTRs), have also been identified.

Another method for detecting variations such as single nucleotide polymorphisms is by the invader assay (U.S. Pat. No. 5,846,717). In this method, a nucleic acid cleavage structure is formed on a target sequence cleaved in a site-specific manner. The 5′ nuclease activity of a variety of enzymes can be used to cleave the target-dependent cleavage structure, thereby indicating the presence of specific nucleic acid sequences or specific variations thereof. As indicated by the commercial supplier of this technology (Third Wave Technologies, Madison, Wis.), no PCR amplification is required.

Denaturing high performance liquid chromatography (DHPLC) for separating heteroduplex (double-stranded nucleic acid molecules having less than 100% sequence complementarity) and homoduplex (double-stranded nucleic acid molecules having 100% sequence complementarity) nucleic acid samples (e.g., DNA or RNA) in a mixture is described in U.S. Pat. Nos. 5,795,976; 6,287,822; and 6,379,889. In the separation method, a mixture containing both heteroduplex and homoduplex nucleic acid samples is applied to a stationary reversed phase support. The sample mixture is then eluted with a mobile phase containing an ion-pairing reagent and an organic solvent. Sample elution is carried out under conditions effective to at least partially denature the duplexes and results in the separation of the heteroduplex and homoduplex molecules. Also disclosed is the amplification of homoduplex and heteroduplex molecules using the polymerase chain reaction. The amplified DNA molecules are denatured and renatured to form a mixture of heteroduplex and homoduplex molecules prior to separating the molecules. In the instant invention, DHPLC is a preferred analytical method and will be further described hereinbelow.

In certain aspects, the instant invention concerns chromatographic separation of DNA fragments for analysis of alterations in DNA. Recently, a chromatographic method called ion-pair reverse-phase high performance liquid chromatography (IP-RP-HPLC), also referred to as Matched Ion Polynucleotide Chromatography (MIPC), was introduced to effectively separate mixtures of double stranded polynucleotides, in general and DNA, in particular, wherein the separations are based on base pair length (Huber, et al., Chromatographia 37:653 (1993); Huber, et al., Anal. Biochem. 212:351 (1993); U.S. Pat. Nos. 5,585,236; 5,772,889; 5,972,222; 5,986,085; 5,997,742; 6,017,457; 6,030,527; 6,056,877; 6,066,258; 6,210,885; and U.S. patent application Ser. No. 09/129,105 filed Aug. 4, 1998.

“Reversed phase support” refers to a stationary support (including the base material and any chemically bonded phase) for use in liquid chromatography, particularly high performance liquid chromatography (HPLC), which is less polar (e.g., more hydrophobic) than the starting mobile phase.

“Ion-pair (IP) chromatography” refers to a chromatographic method for separating samples in which some or all of the sample components contain functional groups which are ionized or are ionizable. Ion-pair chromatography is typically carried out with a reversed phase column in the presence of an ion-pairing reagent.

“Ion-pairing reagent” is a reagent which interacts with ionized or ionizable groups in a sample to improve resolution in a chromatographic separation. An “ion-pairing agent” refers to both the reagent and aqueous solutions thereof. An ion-pairing agent is typically added to the mobile phase in reversed phase liquid chromatography for optimal separation. The concentration and hydrophobicity of an ion-pairing agent of choice will depend upon the number and types (e.g., cationic or anionic) of charged sites in the sample to be separated.

“Primer” refers to an oligonucleotide, whether occurring naturally as in a purified restriction digest or produced synthetically, which is capable of acting as a point of initiation of synthesis when placed under conditions in which synthesis of a primer extension product that is complementary to a target nucleic acid strand is induced, i.e., in the presence of nucleotides and an agent for polymerization (such as a DNA polymerase) and at a suitable temperature and pH. The primer is preferably single stranded for maximum efficiency in amplification. Preferably, the primer is an oligodeoxyribonucleotide. The primer must be sufficiently long to prime the synthesis of extension products (referred to herein as “PCR products” and “PCR amplicons”) in the presence of the polymerization agent.

The term “hybridization” refers to a process of heating and cooling a double stranded DNA (dsDNA) sample, e.g., heating to 95° C. followed by slow cooling. The heating process causes the DNA strands to denature. Upon cooling, the strands re-combine, or re-anneal, into duplexes.

In preparing a set of DNA fragments for analysis by DHPLC, it is usually assumed that all of the fragments have the same length since they are generated using the same set of PCR primers. It is further usually assumed that the fragments are eluted under essentially the same conditions of temperature and solvent gradient. The pattern or shape of the chromatographic separation profile consists of peaks representing the detector response as various species elution during the separation process. The profile is determined by, for example, the number, height, width, symmetry and retention time of peaks. Other patterns can be observed, such as 3 or 2 peaks. The profile can also include poorly resolved shoulders. The shape of the profile contains useful information about the nature of the sample. The pattern or shape of the resulting chromatogram will be influenced by the type and location of the mutation. Each mutation (e.g. single nucleotide polymorphism (SNP)) has a corresponding elution profile, or signature, at a given set of elution conditions of temperature and gradient.

Detection of alterations (i.e. mutations) in DNA requires a highly sensitive, reproducible and accurate analytical method. The design of polymerase chain reaction (PCR) primers used to amplify DNA samples which are to be analyzed for the presence of alterations is an important factor contributing to accuracy, sensitivity and reliability of mutation detection. The design of primers specifically for the purpose of enhancing and optimizing mutation detection by DHPLC is disclosed in U.S. Pat. No. 6,287,822, the PCT publication WO9907899, by Xiao et al. (Human Mutation 17:439-474 (2001) and by Kuklin et al., (Genet. Test. 1:201-206 (1998).

Detection of alterations in dsDNA using DHPLC is more reliable and accurate if the alteration is located within a section having a narrow midpoint temperature range. An example of such a section is the constant melting domain as described by Lerman et al. (Meth. Enzymol. 155:482 (1987)).

Generally, a fragment, such as an exon, will contain sample sequences, or sections, having different melting temperatures, but which have a narrow range of variation within any one section.

When the sequence of a DNA fragment to be amplified by PCR is known, commercially available software can be used to design primers which will produce either the whole fragment, or any section, within the fragment. The primers for use in DHPLC are preferably selected to amplify a section of the target fragment in which the bases have a narrow range of melting point. For example, the range can be less that about 15° C.

In preparing a sample for DHPLC analysis, a selected section of a target DNA fragment can be amplified by PCR using both forward and reverse primers which flank the first and second ends of the section. The amplification product is then hybridized with corresponding reference double stranded DNA prior to analysis. In the instant invention, the reference DNA preferably consists of the sequence of the tester strain that corresponds to the amplicon obtained from the PCR amplification of DNA obtained from the second stage incubation. If there is a back-mutation at the site of the histidine operon, then hybridization of the reference fragment with the amplicon will generate heteroduplex molecules. These can be detected using a variety of methods, including DHPLC, as described herein.

Another aspect of the invention concerns the amplification of a region of cellular DNA that includes the site of the preexisting mutation. The amplification involves nucleic acid amplification procedures, such as PCR, which involve chain elongation by a DNA polymerase. There are a variety of different PCR techniques which utilize DNA polymerase enzymes, such as Taq polymerase. See PCR Protocols: A Guide to Methods and Applications. (Innis, M, Gelfand, D., Sninsky, J. and White, T., eds.), Academic Press, San Diego (1990) for detailed description of PCR methodology. PCR is also described in detail in U.S. Pat. No. 4,683,202 to Mullis (1987); Eckert et al., The Fidelity of DNA polymerases Used In The Polymerase Chain Reactions, McPherson, Quirke, and Taylor (eds.), “PCR: A Practical Approach”, IRL Press, Oxford, Vol. 1, pp. 225-244; Current Protocols in Molecular Biology, Ausubel et al. eds. John Wiley & Sons (1995), Chapter 15; and Andre, et. al., GENOME RESEARCH, Cold Spring Harbor Laboratory Press, pp. 843-852 (1977).

In a typical PCR protocol, a target nucleic acid, two oligonucleotide primers (one of which anneals to each strand), nucleotides, polymerase and appropriate salts are mixed and the temperature is cycled to allow the primers to anneal to the template, the DNA polymerase to elongate the primer, and the template strand to separate from the newly synthesized strand. Subsequent rounds of temperature cycling allow exponential amplification of the region between the primers.

There are a variety of different DNA polymerase enzymes that can be used in PCR, although proof-reading polymerases are preferred. DNA polymerases useful in the present invention may be any polymerase capable of replicating a DNA molecule. Preferred DNA polymerases are thermostable polymerases, which are especially useful in PCR. Thermostable polymerases are isolated from a wide variety of thermophilic bacteria, such as Thermus aquaticus (Taq), Thermus brockianus (Tbr), Thermus flavus (Tfl), Thermus ruber (Tru), Thermus thermophilus (Tth), Thermococcus litoralis (Tli) and other species of the Thermococcus genus, Thermoplasma acidophilum (Tac), Thermotoga neapolitana (Tne), Thermotoga maritima (Tma), and other species of the Thermotoga genus, Pyrococcus furiosus (Pfu), Pyrococcus horikoshii (Pho), Pyrococcus woesei (Pwo) and other species of the Pyrococcus genus, Bacillus sterothermophilus (Bst), Sulfolobus acidocaldarius (Sac) Sulfolobus solfataricus (Sso), Pyrodictium occultum (Poc), Pyrodictium abyssi (Pab), and Methanobacterium thermoautotrophicum (Mth), and mutants, variants or derivatives thereof.

Several DNA polymerases are known in the art and are commercially available (e.g., from Boehringer Mannheim Corp., Indianapolis, Ind.; Life Technologies, Inc., Rockville, Md.; New England Biolabs, Inc., Beverley, Mass.; Perkin Elmer Corp., Norwalk, Conn.; Pharmacia LKB Biotechnology, Inc., Piscataway, N.J.; Qiagen, Inc., Valencia, Calif.; Stratagene, La Jolla, Calif.; Transgenomic, Omaha, Nebr.). Preferably the thermostable DNA polymerase is selected from the group of Taq, Tbr, Tfl, Tru, Tth, Tli, Tac, Tne, Tma, Tih, Tfi, Pfu, Pwo, Kod, Bst, Sac, Sso, Poc, Pab, Mth, Pho, ES4, VENT™, DEEPVENT™, PFUTurbo™, AmpliTaq®, and active mutants, variants and derivatives thereof. It is to be understood that a variety of DNA polymerases may be used in the present invention, including DNA polymerases not specifically disclosed above, without departing from the scope or preferred embodiments thereof.

When retard samples are to be analyzed using DHPLC, the PCR preferably utilizes buffers and other solutions that are compatible with DHPLC analysis, as described in U.S. patent application Ser. No.10/126,848, filed Apr. 19, 2002. The PCR buffers, enzymes preparations, and other solutions minimize, or preferably exclude, BSA, mineral oil, formamide, polyethylene glycol, detergents such as Triton X-100, NP40, Tween 20, sodium dodecyl sulfate and sodium lauryl sulfate. Other reagents, such as those commonly used in the purification of DNA, such as proteases, solvents, nucleases, phenol, guanidinium, etc., are preferably removed in a final ethanol precipitation and wash step prior to PCR. Excess EDTA, isopropanol, or iso-amyl alcohol are also preferably removed. Examples of suitable proof reading enzyme preparations includes Pho polymerase (available as Optimase™ polymerase (Transgenomic) and AccuType™ DNA polymerase (Stratagene).

In a typical PCR protocol, a target nucleic acid, two oligonucleotide primers (one of which anneals to each strand), nucleotides, polymerase and appropriate salts are mixed and the temperature is cycled to allow the primers to anneal to the template, the DNA polymerase to elongate the primer, and the template strand to separate from the newly synthesized strand. Subsequent rounds of temperature cycling allow exponential amplification of the region between the primers.

Oligonucleotide primers useful in the present invention may be any oligonucleotide of two or more nucleotides in length. Preferably, PCR primers are about 15 to about 30 bases in length, and are not palindromic (self-complementary) or complementary to other primers that may be used in the reaction mixture. Oligonucleotide primers are oligonucleotides used to hybridize to a region of a target nucleic acid to facilitate the polymerization of a complementary nucleic acid. Any primer may be synthesized by a practitioner of ordinary skill in the art or may be purchased from any of a number of commercial venders (e.g., from Boehringer Mannheim Corp., Indianapolis, Ind.; New England Biolabs, Inc., Beverley, Mass.; Pharmacia LKB Biotechnology, Inc., Piscataway, N.J.). It will be recognized that the PCR primers can include covalently attached groups, such as fluorescent tags. U.S. Pat. Nos. 6,210,885 and 6,379,889 describe the use of such tags in mutation detection by DHPLC, and include descriptions of multiplex analysis. It is to be understood that a vast array of primers may be useful in the present invention, including those not specifically disclosed herein, without departing from the scope or preferred embodiments thereof.

When analysis is performed using DHPLC in the present invention, the length of the amplicon is preferably between about 100 and about 700 bp, and more preferably between about 150 bp and about 500 bp.

Buffering agents and salts are used in the PCR buffers and storage solutions of the present invention to provide appropriate stable pH and ionic conditions for nucleic acid synthesis, e.g., for DNA polymerase activity, and for the hybridization process. A wide variety of buffers and salt solutions and modified buffers are known in the art that may be useful in the present invention, including agents not specifically disclosed herein. Preferred buffering agents include, but are not limited to, TRIS, TRICINE, BIS-TRICINE, HEPES, MOPS, TES, TAPS, PIPES, CAPS. Preferred salt solutions include, but are not limited to solutions of; potassium acetate, potassium sulfate, ammonium sulfate, ammonium chloride, ammonium acetate, magnesium chloride, magnesium acetate, magnesium sulfate, manganese chloride, manganese acetate, manganese sulfate, sodium chloride, sodium acetate, lithium chloride, and lithium acetate.

In DHPLC, the length and diameter of the separation column, as well as the system mobile phase pressure and temperature, and other parameters, can be varied. An increase in the column diameter was found to increase resolution of polynucleotide fragments in IP-RP-HPLC and DHPLC (U.S. Pat. No. 6,372,142; WO 01/19485). Size-based separation of DNA fragments can also be performed using batch methods and devices as disclosed in U.S. Pat. Nos. 6,265,168; 5,972,222; and 5,986,085.

In DHPLC, the mobile phase typically contains an ion-pairing agent (i.e. a counter ion agent) and an organic solvent. Ion-pairing agents for use in the method include lower primary, secondary and tertiary amines, lower trialkylammonium salts such as triethylammonium acetate and lower quaternary ammonium salts. Typically, the ion-pairing reagent is present at a concentration between about 0.05 and 1.0 molar. Organic solvents for use in the method include solvents such as methanol, ethanol, 2-propanol, acetonitrile, and ethyl acetate.

In one embodiment of DHPLC, the mobile phase for carrying out the separation contains less than about 40% by volume of an organic solvent and greater than about 60% by volume of an aqueous solution of the ion-pairing agent. In a preferred embodiment, elution is carried out using a binary gradient system.

Partial denaturation of heteroduplex molecules can be carried out in a variety of ways such as alteration of pH or salt concentration, use of denaturing agents, or elevation in temperature. Temperatures for carrying out the separation are typically between about 50° and 70° C. and preferably between about 55° and 65° C. The preferred temperature is sequence dependent. In carrying out a separation of GC-rich heteroduplex and homoduplex molecules, for example, a higher temperature is preferred.

A variety of liquid chromatography systems are available that can be used for conducting DHPLC. These systems typically include software for operating the chromatography components, such as pumps, heaters, mixers, fraction collection devices, injector. Examples of software for operating a chromatography apparatus include HSM Control System (Hitachi), ChemStation (Agilent), VP data system (Shimadzu), Millennium32 Software (Waters), Duo-Flow software (Bio-Rad), and Star workstation (Varian). Examples of preferred liquid chromatography systems for carrying out DHPLC include the WAVE® DNA Fragment Analysis System (Transgenomic) and the Varian ProStar Helix™ System (Varian).

In carrying out DHPLC analysis, the operating temperature and the mobile phase composition can be determined by trial and error. However, these parameters are preferably obtained using software. Computer software that can be used in carrying out DHPLC is disclosed in the following patents and patent applications: U.S. Pat. Nos. 6,287,822; 6,197,516; U.S. patent application Ser. No. 09/469,551 filed Dec. 22, 1999; and in WO0146687 and WO0015778. Examples of software for predicting the optimal temperature for DHPLC analysis are disclosed by Jones et al. in Clinical Chem. 45:113-1140 (1999) and in the website having the address of http://insertion.stanford.edu/melt.html. Examples of a commercially available software include WAVEMaker® software and Navigator™ software (Transgenomic).

The IP-RP-HPLC retention times of double stranded DNA fragments can be predicted using software such as Wavemaker™ software (Transgenomic) or Star workstation software (Varian). These programs allow prediction of the retention time based on the length of a DNA fragment for a given set of elution conditions (U.S. Pat. Nos. 6,287,822 and 6,197,516; and in U.S. patent application Ser. No. 09/469,551 filed Dec. 22, 1999; and PCT publications WO99/07899 and WO 01/46687).

Suitable separation media for performing DHPLC are described in the following U.S. patents and patent applications: U.S. Pat. Nos. 6,379,889; 6,056,877; 6,066,258; 5,453,185; 5,334,310; U.S. patent application Ser. No. 09/493,734 filed Jan. 28, 2000; U.S. patent application Ser. No. 09/562,069 filed May 1, 2000; and in the following PCT applications: WO98/48914; WO98/48913; PCT/US98/08388; PCT/US00/11795. Examples of suitable media include separation beads and monolithic rods. An example of a suitable column based on a polymeric stationary support is the DNASep® column (Transgenomic). Examples of suitable columns based on a silica stationary support include the Microsorb Analytical column (Varian and Rainin) and “ECLIPSE dsDNA” (Hewlett Packard, Newport, Del.).

IP-RP-HPLC is preferably conducted using a separation medium that is substantially free of metal contaminants or other contaminants that can bind nucleic acids or that can interfere with the separation. Preferred beads and monoliths have been produced under conditions where precautions have been taken to substantially eliminate any multivalent cation contaminants (e.g. Fe(III), Cr(III), or colloidal metal contaminants), including a decontamination treatment, e.g., an acid wash treatment. Very pure, non-metal containing materials are preferred in the production of the beads in order to minimize the metal content of the resulting beads.

In addition to the separation medium being substantially metal-free, to achieve optimum peak separation the separation column and all process solutions held within the column or flowing through the column are preferably substantially free of multivalent cation contaminants (e.g. Fe(III), Cr(III), and colloidal metal contaminants). As described in U.S. Pat. Nos. 5,772,889, 5,997,742 and 6,017,457, this can be achieved by supplying and feeding solutions that enter the separation column with components that have process solution-contacting surfaces made of material which does not release multivalent cations into the process solutions held within or flowing through the column, in order to protect the column from multivalent cation contamination. The process solution-contacting surfaces of the system components are preferably material selected from the group consisting of titanium, coated stainless steel, passivated stainless steel, and organic polymer.

Trace levels of multivalent cations anywhere in the solvent flow path can cause a significant deterioration in the resolution of the separation after multiple uses of an IP-RP-HPLC column. This can result in increased cost caused by the need to purchase replacement columns and increased downtime. Therefore, effective measures are preferably taken to prevent multivalent metal cation contamination of the separation system components, including separation media and mobile phase contacting. These measures include, but are not limited to, washing protocols to remove traces of multivalent cations from the separation media and installation of guard cartridges containing cation capture resins, in line between the mobile phase reservoir and the IP-RP-HPLC column. These, and similar measures, taken to prevent system contamination with multivalent cations have resulted in extended column life and reduced analysis downtime.

For additional protection, multivalent cations in mobile phase solutions and sample solutions entering the column can be removed by contacting these solutions with multivalent cation capture resin before the solutions enter the column to protect the separation medium from multivalent cation contamination. The multivalent capture resin is preferably cation exchange resin and/or chelating resin.

There are two places where multivalent-cation-binding agents, e.g., chelators, can be used in DHPLC separations. In one embodiment, these binding agents can be incorporated into a solid through which the mobile phase passes. Contaminants are trapped before they reach places within the system that can harm the separation. In these cases, the functional group is attached to a solid matrix or resin (e.g., a flow-through cartridge, usually an organic polymer, but sometimes silica or other material). The capacity of the matrix is preferably about 2 mequiv./g. An example of a suitable chelating resin is available under the trademark CHELEX 100 (Dow Chemical Co.) containing an iminodiacetate functional group.

In another embodiment, the multivalent cation-binding agent can be added to the mobile phase. The binding functional group is incorporated into an organic chemical structure. The preferred multivalent cation-binding agent fulfills three requirements. First, it is soluble in the mobile phase. Second, the complex with the metal is soluble in the mobile phase. Multivalent cation-binding agents such as EDTA fulfill this requirement because both the chelator and the multivalent cation-binding agent-metal complex contain charges, which makes them both water-soluble. Also, neither precipitate when acetonitrile, for example, is added. The solubility in aqueous mobile phase can be enhanced by attaching covalently bound ionic functionality, such as, sulfate, carboxylate, or hydroxy. A preferred multivalent cation-binding agent can be easily removed from the column by washing with water, organic solvent or mobile phase. Third, the binding agent must not interfere with the chromatographic process.

In the instant invention, after the second stage incubation, DNA can be extracted from the cells (such as obtained after the two-step process or the one-step process) using conventional methods. However, in another aspect of the instant invention, Applicants have observed that the application of high temperature lyses the tester strain cells and releases the DNA, and that there is no need to extract the DNA from the bacteria prior to PCR. In a preferred embodiment of this method, the second stage medium containing tester strain cells (i.e. from the 1 ul aliquot) is heated to 95° C. for 15 minutes prior to the beginning of the PCR cycle. This heating step can be at 95° C. for 5 to 20 minutes, and preferably for 15 minutes, prior to the beginning of the PCR cycle. This procedure allows setting up the PCR reactions at room temperature and does not require the use of wax and other barrier methodologies to sequester essential reaction cofactors or the use antibodies to inactivate Taq polymerase to achieve “Hot start” PCR conditions, ensuring specific product formation.

After the PCR, the DNA can be quantified, such as by UV absorption measurement. An amount (e.g. 1:1) of “exogenous” reference double stranded DNA can be added, and the mixture subjected to hybridization. The exogenous reference DNA is the same length as the amplified DNA, and has the original sequence of the tester strain used. The hybridized sample is then analyzed, such as by DHPLC. Disadvantages of this procedure are the requirement to quantify the DNA level after the PCR and the requirement to add reference DNA.

In an additional aspect of the present invention, Applicants have devised a method which avoids the need to add exogenous reference DNA prior to the hybridization step in DHPLC or other methods. This can be achieved, as indicated herein, by the inclusion of LB or media components that favor limited tester strain growth, to promote the enrichment of revertants to a level detectable by the mutation detection methodology employed. DHPLC can reliably detect a component if present at greater than about 5 to 10% in the mixed population. The more sensitive the mutation detection method, the less revertant enrichment time is needed to achieve the desired ratio level of revertant to tester strain in an incubation, which can provide a faster assay. The tester strain population provides an “endogenous” reference DNA during the PCR process. An advantage of this method is that no “exogenous” reference DNA need be added. At the end of the PCR, the amplified products are denatured and re-annealed by a hybridization procedure that can be included in the PCR protocol. Heteroduplexes will form if any fragments containing back-mutation were present during the PCR. If the back-mutations involve one or more base addition or deletion, IP-RP-HPLC can be used in a sizing based application (non-denaturing elution conditions) and would not require the denaturation and reannealing step following PCR.

In an additional aspect of the present invention, Applicants have devised a method which avoids the need to add exogenous reference DNA prior to the hybridization step. In an embodiment of this method, the first stage incubation includes a small but known amount of LB medium. This allows growth of an amount of the tester strain until the LB medium is exhausted. The amount of growth reaches a fixed limit that determined by the small amount of histidine that is present in the LB medium. During the second stage incubation, the fixed amount of tester strain does not propagate, because the medium lacks histidine. However, this fixed amount of tester strain provides a quantity of “endogenous” reference DNA. The quantity is determined by the amount of LB broth in the first stage incubation, and can be adjusted by selecting the amount of LB broth in the first stage. A preferred amount of LB is about 1% by volume. This “endogenous” reference DNA is amplified during the PCR along with the DNA from any revertant cells. An advantage of this method is that no “exogenous” reference DNA need be added. At the end of the PCR, the amplified products are denatured and re-annealed by a hybridization procedure that can be included in the PCR protocol. Heteroduplexes will form if any fragments containing back-mutation were present during the PCR.

In some embodiments of the present invention, no PCR amplification is required in order to detect mutations in DNA. For example, when TA102 is used as the tester strain, the site of the preexisting mutation is located on a plasmid (pAQ1). The plasmid is replicated during the second stage incubation and can be isolated thereafter by conventional methods. The plasmid DNA can be cleaved using restriction enzymes, and the restriction fragment containing the target site can be hybridized with same-length reference DNA. DNA. The use of certain mutation detection technologies, such as the invader assay, do not require PCR amplification.

In one embodiment of the invention, a dose-response curve can be obtained, showing the concentration of test substance on one axis, and a value indicating the level of the back-mutation associated with reversion (such as area-under-the-curve for the heteroduplexes) on the other axis. When DHPLC is used for analysis, a “DHPLC-Specific Mutagenic Activity” is defined herein to include the slope of the linear portion of a dose response curve, and is preferably reported, as AUC/ug. The dose response curve can be used to establish background mutation level to assess the mutagenic potential of test compounds.

Another aspect of the present invention concerns the classification of test substances as mutagens. In the traditional AMES test, a test substance is considered to give a positive mutagenic response if the number of revertants (i.e. colonies per plate) after exposure to the substance is at least twice the background (in colonies per plate). A minimum of five dose levels covering a range of three logs are typically used with results duplicated. The background is the number of revertants observed when the test strain is exposed under identical conditions but in the presence of vehicle without the test substance (i.e. a negative control). Examples of vehicle include water, DMSO, ethanol, acetone, ether, and corn oil.

In the present invention, a “background” or “background result” is generally defined to include the result obtained from a negative control. The result can depend on the particular method of detection that is being employed. In the invention, genetic back-mutations occurring at the preexisting mutation site, or nearby (i.e. within about 200 base pairs on either side of the preexisting mutation), are detected at the nucleic acid level. A background result is the occurrence of such back-mutations in a negative control.

A result can have different components. For example, strain TA98 has at least three reversion sites within a 50 base pair stretch around the preexisting mutation, and mutations can occur at one or more of these sites to give a back-mutation.

Controls including known mutagens (positive controls) are preferably performed to demonstrate the sensitivity of the selected test strain to a known direct-acting mutagen and the capability of the activation system (when included) to convert a promutagen to a mutagen.

In one embodiment of a method for determining mutagenicity, the mutagenicity of a test substance can be determined by comparing the test substance induced result to the background result over a pre-selected dose range. For example, a test strain can be exposed to varying doses of test substance, and a pot of response vs. dose obtained. In an example of this embodiment, when DHPLC is used as a detection method, a test substance to be evaluated positive, must cause a dose-responsive increase in the mean in the AUC values due to heteroduplex (the combined AUC if there is more than one heteroduplex peak) of at least one tester strain with or without activation. Data sets are judged positive if the increase in mean AUC values of the dose response is equal to or greater than two times the mean vehicle control value. Triplicate exposures are made at each dose, and the entire dose-response experiment is run in duplicate. Data are subjected to conventional statistical analysis (such as described in Jacobson-Kram, et al (2000)). (The criteria level of a 2-fold increase is arbitrary. Other values for the criteria level can be used.)

Another embodiment of a method for determining mutagenicity includes a “binary” analysis in which a positive or negative mutagenic response is determined. In an example of this method, a first number of incubations of tester cells after exposure to test substance are performed. The number of positive responses as determined by DHPLC analysis is obtained. The mean (test result) and standard deviation are determined. The analyses are repeated at various doses of test substance (e.g. concentrations from 0.1 to 1000 ug/ml) and incubations in the presence and absence of S9 are performed. A second number of negative controls are performed, and the number of positive responses for each incubation is determined. The mean (background result) and standard deviation are determined. The test substance is classified as a mutagen if the mean test result is greater than two times the mean background result at all concentrations of test substance. (The criteria level of a 2-fold increase is arbitrary. Other values for the criteria level can be used.) The number of test and background incubations required can be selected based on the background result. The number of replicates required in order to achieve a pre-selected level of confidence can be calculated by conventional statistical methods. Preferably, the number of test incubations is 2-3.5 times the background frequency. For example, if the background results in 1 or less positive results per 10 repeat incubations, a triplicate incubation is sufficient for mutagenicity testing. If the background result is about 1 to 3 positive results per 10 repeat incubations, then 9 incubations are sufficient for mutagenicity testing.

A “positive response” is defined herein to include the detection of a back-mutation.

As an example of the determination of a positive response when detection is by DHPLC, a tester strain is exposed to a test substance, and the resulting pattern of the DHPLC profile is analyzed. The value of AUC for the heteroduplex peak (or combined peaks) is obtained and the AUC for the homoduplex peak (or combined peaks) is also obtained. If a value for the heteroduplex AUC is obtained that is 5% or greater than the value for the homoduplex AUC, then a positive response is assigned. The limit of detection varies with the detection method. For sequencing-based methods, a 20% or 80% revertant population is needed for detection.

The phrase “statistically significant” is defined herein to include the involvement of parameters such as confidence limits, standard deviations, percentiles, etc. In addition, a boundary such as a 95% confidence limit, can be chosen arbitrarily or by convention. The term “statistical significance” is not limited to any particular method of analysis, or to any particular boundary.

In another aspect of the invention, a plurality of different test strains and all having the same nutritional deficiency (i.e. histidine for the Ames salmonella strains) can be combined in any combination, using the two-sage or one-step method, for each test compound exposure dose, to be simultaneously tested for the presence back-mutant fragments over background. Where the mutation detection technology requires amplification, multiplex PCR can be performed. Multiplex detection is possible by engineering the amplicons to be different in size or by differentially labeling these fragments (e.g., with different fluorophores) (e.g., see U.S. Pat. Nos. 6,210,885, 6,379,889 and PCT publication WO 99/19514). In the case of differential labeling, the appropriate hardware can be used for detection (e.g., for DHPLC a fluorescent detector is required). Strains used for multiplexing include but are not limited to TA97, TA98, TA100, TA102, TA1535, TA1537, and TA1538. The multiplexing detection capability is not limited to DHPLC but can apply to other DNA mutation detection techniques without departing from the scope of the preferred embodiment thereof.

A further aspect of the invention concerns a kit for performing an evaluation of a test compound for mutagenicity. An embodiment of this aspect includes reagents for practicing the method of the invention can be packaged together to form a kit in accordance with the invention. Such a kit can include one or more of the following in packaged combination:

One or more tester strains of Salmonella. These are preferably packaged as lyophilized preparations. In one preferred embodiment the optimal amount of the proper stain or combined strains can be provided in a ready-to-use format (e.g., directly packaged in the reaction vessel). Example of preferred strains include TA98, TA100, TA102, TA104, TA1535, TA1537, TA1538, and TA97.

Reconstitution or growth media. This includes media suitable for use in reconstituting or growing the lyophilized tester strains.

Media to be used for exposure in the two-stage process or for the one-step process.

Optimized for each tester strain, amplification primers specific for amplification of a region of the Salmonella histidine operon. Suitable primers are capable of amplifying regions that contain the preexisting mutation for each strain. Examples of such primer pairs include those that generate amplicons having a length of about 150 base pairs to about 500 base pairs. Primers can be multiplexed if desired for multiplex PCR and detection.

Reference DNA comprising a DNA fragment for use in a hybridization protocol in DHPLC, and corresponding with a region of the histidine operon in an Ames tester strain, the region containing the site of a preexisting mutation for the strain. Both tester strain and revertant fragments can be supplied separately or mixed.

Positive PCR controls for each tester strain. Examples of such controls include cells or purified DNA. The controls would generate, following PCR, a mixture of tester and revertant fragments that produce the characteristic DHPLC pattern observed for back-revertants.

Standard mutagenic compounds to be used as positive controls. Examples of such compounds include, in separate containers, a hydrocarbon mixture derived from petroleum, HC235 extract, azide, daunomycin, hydrazine, methyl methanesulfonic acid, and 2-aminofluorene, mitomycin C, ICR191, 9-amino acrydine, danthron, cyclophosphamide, benzo[a]pyrene, ethyl methanesulfonate and 2-aminofluorene.

S-9 fraction. Examples of suitable S9 includes mammalian liver microsomes, such as obtained from rat, mouse, or hamster. A preferred S9 is Aroclor induced hamster S9.

NADPH generating system.

An antibiotic, such an ampicillin.

Vehicle, such as water or DMSO.

M9 minimal salts.

A DNA polymerase. Examples of suitable polymerases are Pho, Pfu, or Taq polymerase, or mixtures thereof.

A complete protocol describing step-by-step instructions along with appropriate information needed to carry out the test, analyze and interpret results and troubleshoot.

A further aspect of the invention involves a dedicated DHPLC instrument with proper software designed to facilitate analysis, archiving, and troubleshooting of mutagenesis data with proper traceability for GMP/GLP processing. The dedicated instrument can include a conventional DHPLC system, such as the WAVE® or WAVE®-MD system. Another aspect of the invention involves a centralized data management system, such as can be created on the world wide web, where data, methods, and information can be accessed and shared by users worldwide.

The present invention for analyzing test compounds for mutagenicity presents a number of advantages over the traditional Ames test. The analysis time is shortened from 3-5 days to about 1-2 or days or less. All steps can be automated (exposure, enrichment and analysis). A one-to-one correlation exists between exposure dose and results, unlike the one dose one plate result for traditional Ames, with about 200 plates needed per test compound per strain tested. The one-to-one correlation permits higher throughput and greatly simplifies the assay.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All patent applications, patents, and literature references cited in this specification are hereby incorporated by reference in their entirety. In case of conflict or inconsistency, the present description, including definitions, will control. Unless mentioned otherwise, the techniques employed or contemplated herein are standard methodologies well known to one of ordinary skill in the art. The materials, methods and examples are illustrative only and not limiting. All numerical ranges in this specification are intended to be inclusive of their upper and lower limits. Other features of the invention will become apparent in the course of the following descriptions of exemplary embodiments which are given for illustration of the invention and are not intended to be limiting thereof. Procedures described in the past tense in the Examples below have been carried out in the laboratory. Procedures described in the present tense have not yet been carried out in the laboratory, and are constructively reduced to practice with the filing of this application.

### EXAMPLE 1

Evaluation of Mutagenicity of Sodium Azide

Reactions were performed in sterile 1.5 ml microfuge tubes. Each tube contained: In a first stage incubation, 1 ul of overnight culture of TA 1535; 6.5 ul LB; 15 ul H₂O (or S9); 67.5 ul M9 (−histidine+biotin). These four components are added from a master mix to the microfuge tubes. 10 ul of sodium azide solution was added to each tube. The final levels of sodium azide were 0-6 ug. The mixture was incubated 1.5 hours at 37° C. The lids of the tubes were closed, but ventilated by piercing with a needle, for aeration. In a second stage incubation, 800 ul of Selective Enrichment Media was added, and the mixture is incubated for 18 hrs without shaking.

Selective Enrichment Media was prepared in a final volume of 900 ml as follows:

| | | |
|---|---|---|
| M9 50x part A (Qbiogene, | 18 | ml |
| catalog no. 3035-012) | | |
| M9 50x part B- | 18 | ml |
| CSM-his (Qbiogene, catalog | 0.77 | g |
| no. 4510-312) | | |
| 5 M NaCl | 15.3 | ml |
| H₂O | 758 | ml |

The media was autoclaved and cooled to room temperature. 90 ml of autoclaved 20% glucose was added. Prior to use, Biotin (Sigma) was added from a 400× stock (1.25M) to a final concentration of 1×.

A 1 ul aliquot was used in a “Hot start” PCR amplification with a final denaturation/renaturation step followed by analysis by DHPLC. Each dose was processed in quadruplicate.

For TA1535, the following 342 bp amplicon from the hisG gene was prepared:

(SEQ ID NO:1)

CGGTTTAAGCGATGATTCACGAGAATTGCTGGGCCGGTGCGGCATAAAAA
TTAATTTAGACACTCAGCGCCTGATTGGGATGGGGGAAAAGATGCCGATT
GATATGCTGCGCGTGCGTGATGATGACATTGCGGGTCTGGTAATGGATGG
CGTGGTCGATCCCGGTATTATCGGCGAAAACGTGGTGGAAGAAGAGCTAC
TCAACCGCGGCGCACAGGGCGAAGATCCACGGTATTTAACCCTGCGCCGT
CTTGACTTGGGCGGCTGCCGTTTATCGCTGGCAACACCGGTTGAGGAAGC
CTGGGAGGGCCCGGCCGCGCTGGACGGTAAACGTATCGCTAC

(The mutation hot spot is underlined. The central C changes to T in revertants)

In the PCR, the forward primer was CCGTTTAAGCGATGATTCACGA (SEQ ID NO: 2) and the reverse primer was GTAGCGATACGTTTACCGTCCA (SEQ ID NO: 3). The DNA polymerase was Optimase™ polymerase. The PCR conditions were as follows:

| Step | Temp. | | Duration (min) |
|---|---|---|---|
| 1. | 95° C. | | 20:00 |
| 2. | 95° C. | | 00:30 |
| 3. | 64° C. | | 01:00 |
| | −0.5° C. | | |
| | per cycle | | |
| 4. | 72° C. | | 01:00 |
| 5. | go to 2 | 15 | |
| | | times | |
| 6. | 95° C. | | 00:30 |
| 7. | 59° C. | | 01:00 |
| 8. | 72° C. | | 01:00 |
| 9. | go to 6 | 20 | |
| | | times | |
| 10. | 72° C. | | 05:00 |
| 11. | 95° C. | | 04:00 |
| 12. | 95° C. | | 01:00 |
| | −1.5° C. | | |
| | per cycle | | |
| 13. | go to 12 | 48 | |
| | | times | |
| 14. | 4° C. | storage | |

DHPLC analysis was performed using a WAVE® Model 2100A chromatography system (Transgenomic) equipped with a DNASep® cartridge (50 mm×4.5 mm ID). The injection volume was 10 ul. The column temperature was 64° C.

The aqueous mobile phase consisted of Buffer A: 100 mM triethylammonium acetate (TEAA) (pH 7) (Transgenomic), Buffer B: 100 mM TEM in 25% acetonitrile (VWR Scientific) (pH 7), and Buffer D: 75% acetonitrile in water. High purity water used for preparing buffer solutions was obtained using a Milli-Q water system (Millipore, Milford, Mass.).

| Time | % A | % B | % D |
|---|---|---|---|
| 0.0 | 50 | 50 | 0 |
| 0.5 | 45 | 55 | 0 |
| 5.0 | 36 | 64 | 0 |
| 5.1 | 0 | 0 | 100 |
| 5.6 | 0 | 0 | 100 |
| 5.7 | 50 | 50 | 0 |
| 6.6 | 50 | 50 | 0 |

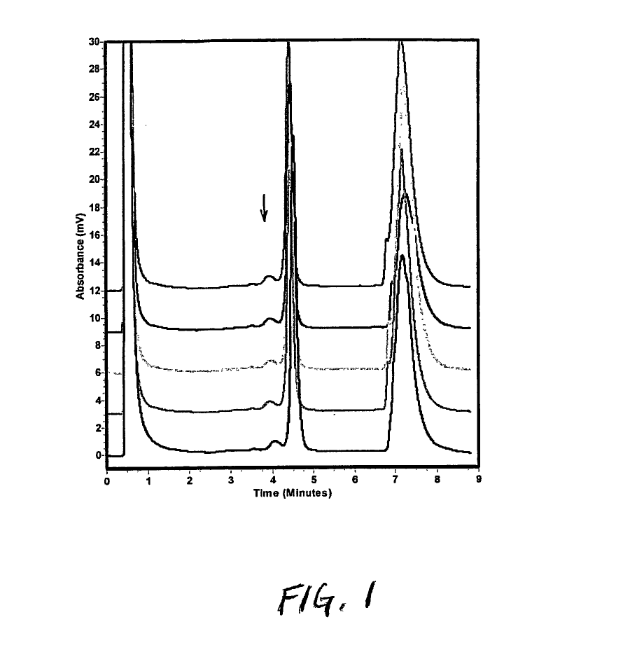

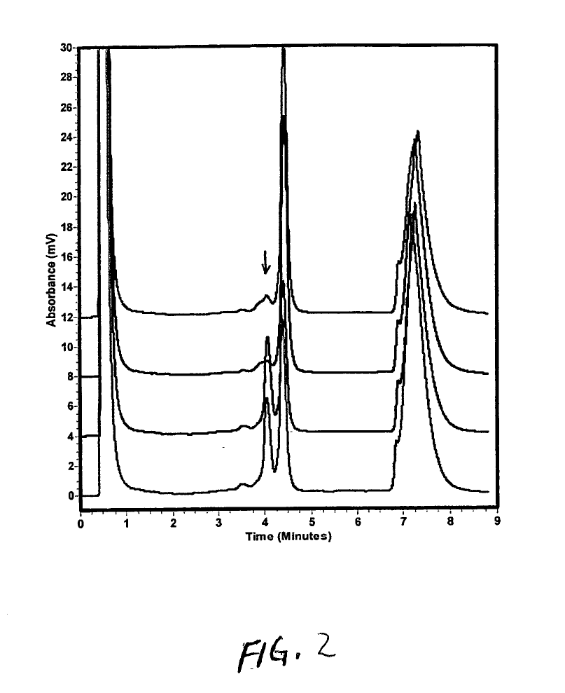

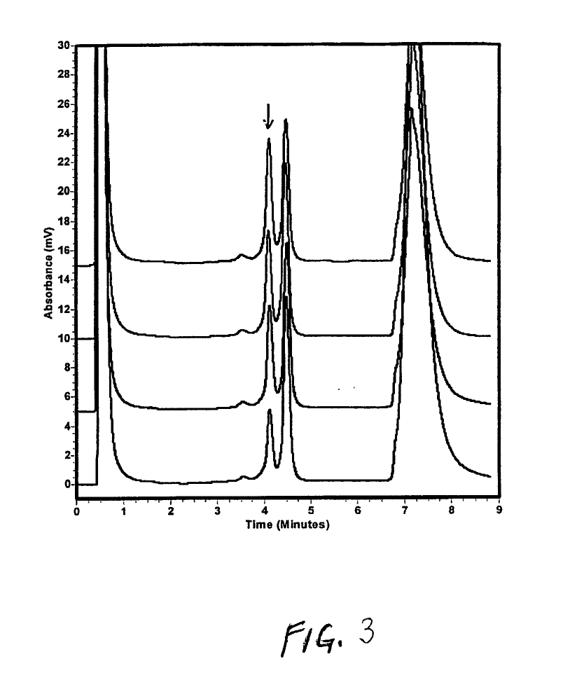

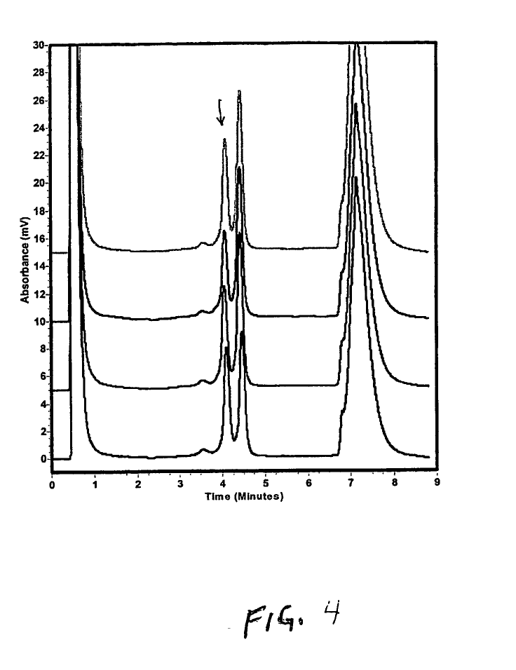

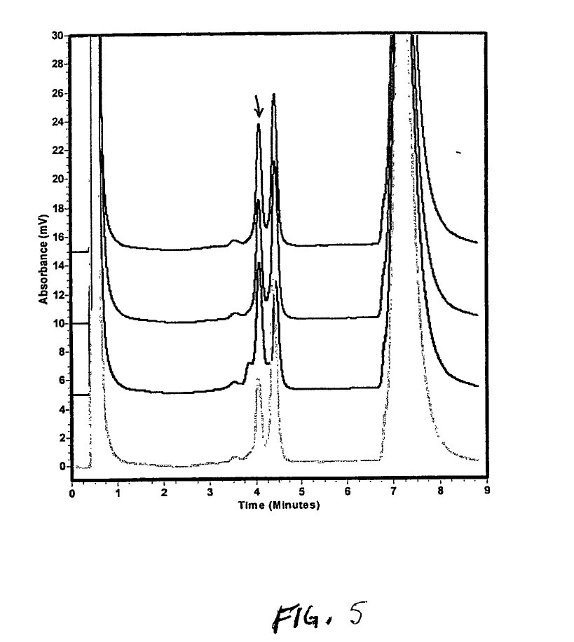

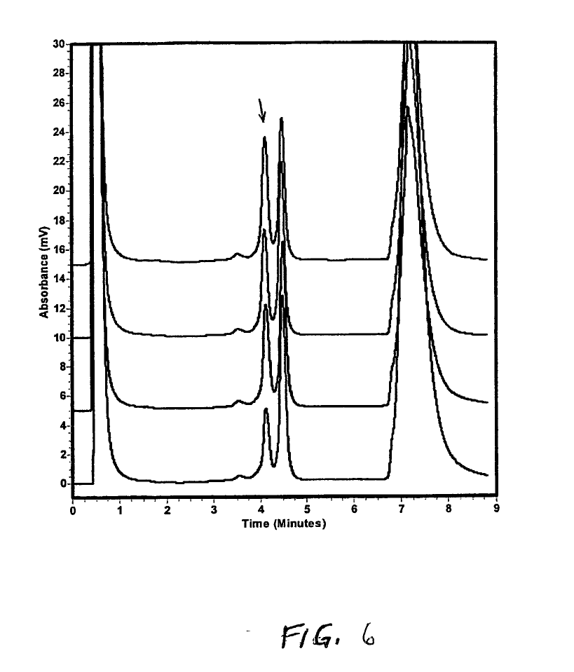

Exposure of TA1535 to sodium azide caused DNA back-mutation at the hot spot. In its absence, no heteroduplex was detected (vehicle control) as shown in FIG. 1. FIGS. 2-6 represent DHPLC chromatographs obtained when cells were exposed to azide levels of 0.08, 0.2, 0.66, 2.0, and 6 ug, respectively.

For comparative purposes, Ames modified plate assay were performed. Tubes were prepared as above, but no Selective Enrichment Media was added. After the 1.5-hour exposure, the incubation was add to 2 ml of melted top agar, containing limited histidine and biotin, equilibrated to 50° C. (MOLTOX catalog no. 26-503) and plated on minimal plates lacking histidine (MOLTOX catalog no.21-400). The plates were incubated 2 days at 37° C. and revertant colonies counted as shown:

| NaN₃ | | | |
|---|---|---|---|
| (exposure | | no. revertant | no. revertant |
| amount) | | colonies | colonies |
| 6 | ug | >100 | >100 |
| 2 | ug | 39 | 66 |
| 0.6 | ug | 15 | 27 |
| 0.2 | ug | 13 | 13 |
| 0.08 | ug | 3 | 5 |
| 0 | | 4 | NA |

### EXAMPLE 2

Dose-Response of UV Treatment on Salmonella Strain TA102

TA102 cells were prepared in a 100 ul first stage incubation as described in Example 1, except 75 ul of overnight culture was obtained and uniformly spread onto a plate, followed by exposure to UV light for 0, 3, 15, or 30 seconds. A second stage incubation was performed using 8 ml of enrichment media solution, followed by PCR.

For TA102, the following 421 bp amplicon from the hisG428 gene was prepared:

(SEQ ID NO:4)

TCCTCAAACGCTACCTCGACCAGAAAGGCGTCTCTTTTAAATCGTGTCTG
TTAAATGGTTCTGTCGAAGTCGCGCCGCGCGCGGGGCTGGCCGACGCTAT
CTGCGATTTGGTCTCTACCGGCGCGACGCTTGAAGGTAAGGGCCTGGGTG
AAGTCGAAGTTATGTACGGGTCTAAAGCCTGTCTGATTCAGCGCGACGGT
GAGATGGCACAGAGCAAGCAAGAGCTGATCGATAAATTGGTGACCGGTAT
TCAGGGCGTGATTCAGGCGCGCGAATCGAAATACATCATGATGGAGGGGC
GAAGTGAACGCCTGGAAGAGGTTATCGCCCTGCTGCCAGGCGCCGAAAGG
CCGACAATTCTGCCGCTGGCAGGCGAGCAACAGCGCGTGGCGATGGACAT
GGTCAGCAGCGAAACGTTGTT

(The mutation hot spot is underlined. The first C in histidine-dependant wild type changes to T in the revertant)

In the PCR, the forward primer was TCCTCAAACGCTACCTCGACCA (SEQ ID NO: 4), and the reverse primer was AACAACGTTTCGCTGCTGACCA (SEQ ID NO: 5). PCR conditions were as follows:

| | | | Duration |
|---|---|---|---|
| Stage | Temp. | | (min) |
| 1. | 95° C. | | 20:00 |
| 2. | 95° C. | | 00:30 |
| 3. | 66° C. | | 01:00 |
| | −0.5° C. | | |
| | per cycle | | |
| 4. | 72° C. | | 01:00 |
| 5. | go to 2 | 15 | |
| | | times | |
| 6. | 95° C. | | 00:30 |
| 7. | 61° C. | | 01:00 |
| 8. | 72° C. | | 01:00 |
| 9. | go to 6 | 20 | |
| | | times | |
| 10. | 72° C. | | 05:00 |
| 11. | 95° C. | | 04:00 |
| 12. | 95° C. | | 01:00 |
| | −1.5° C. | | |
| | per cycle | | |
| 13. | go to 12 | 48 | |
| | | times | |
| 14. | 4° C. | | storage |

DHPLC analysis was performed using a WAVE System as described in Example 1, but at a column temperature of 66° C. and using the following mobile phase gradient:

| Time | % A | % B | % D |
|---|---|---|---|
| 0.0 | 48 | 52 | 0 |
| 0.5 | 43 | 57 | 0 |
| 5.0 | 34 | 66 | 0 |
| 5.1 | 0 | 0 | 100 |
| 5.6 | 0 | 0 | 100 |
| 5.7 | 48 | 52 | 0 |
| 6.6 | 48 | 52 | 0 |

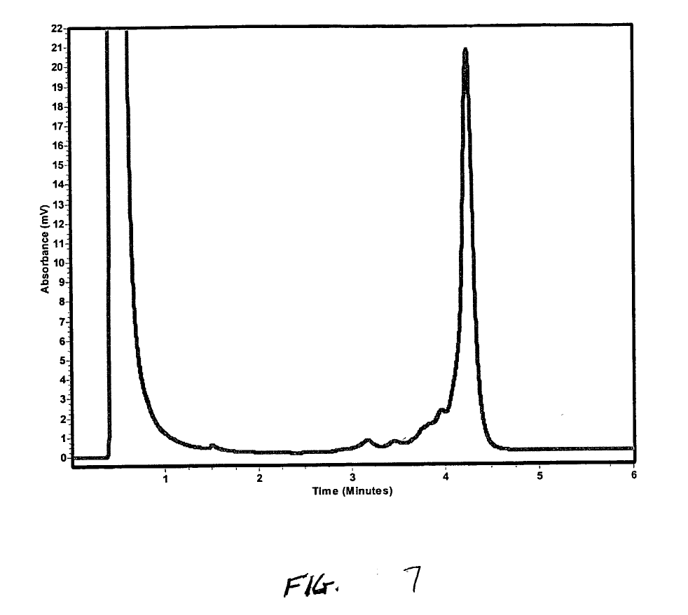

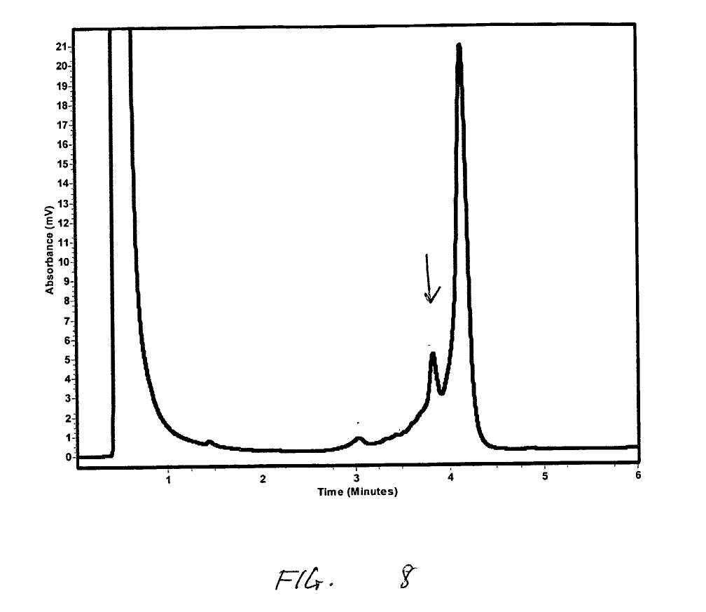

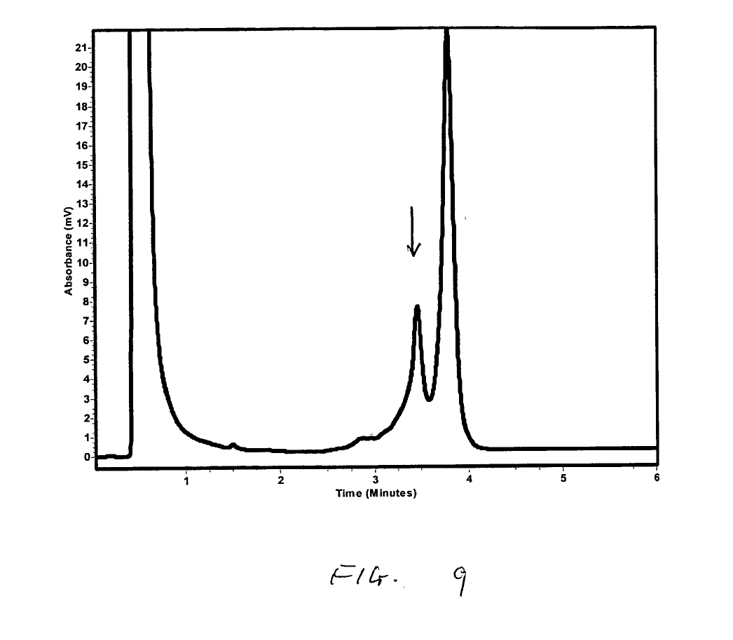

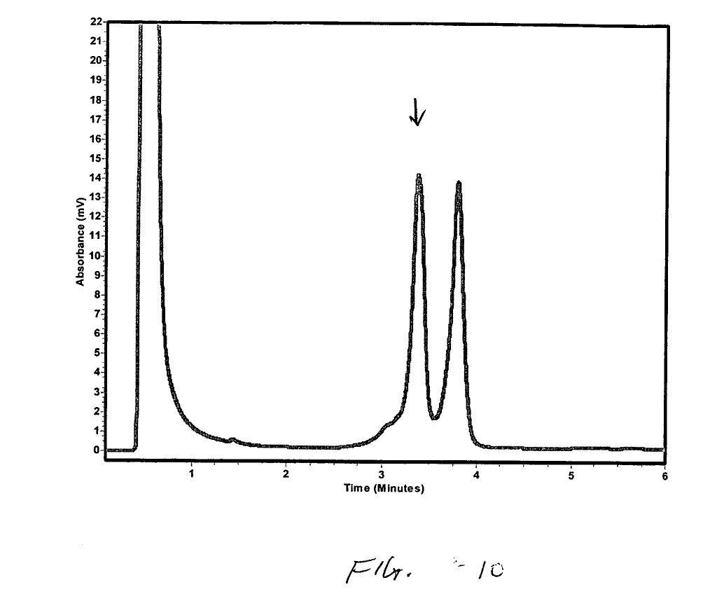

FIGS. 7-10 show chromatographic elution profiles obtained from DHPLC analysis of DNA from Salmonella cells exposed to UV light. The exposure time was 0 min in FIG. 7, 3 sec. in FIG. 8, 15 sec. in FIG. 9, and 30 sec. FIG. 10. There is an increase in the peak height and the area-under-the-curve (AUC) due to the heteroduplex (arrow in each figure) with increasing exposure time.

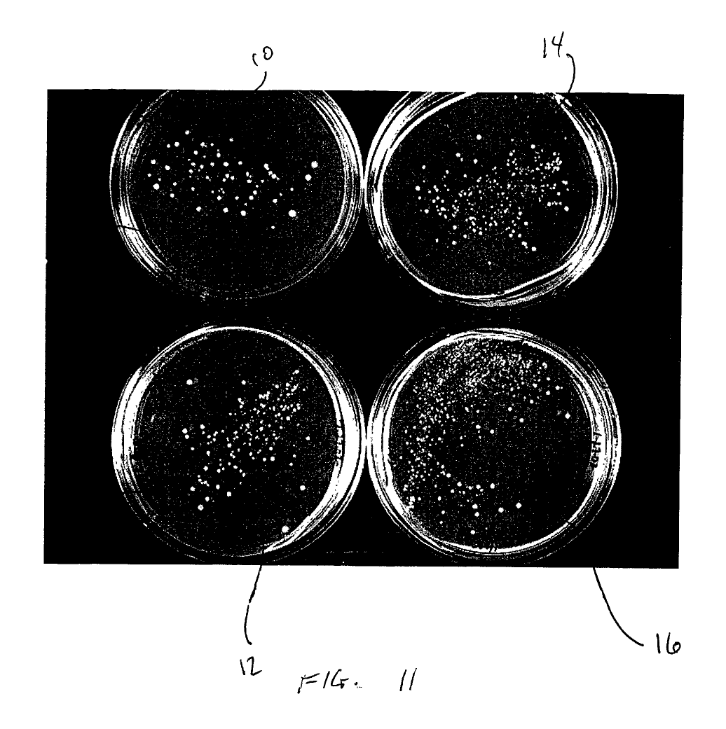

For comparison, an Ames modified plate assay was performed as described in Example 1. FIG. 11 illustrates growth of Salmonella colonies on an agar plate after exposure to UV light. The plates were exposed to UV light for the following exposure periods: plate 10, 0 sec; plate 12, 10 sec; plate 14, 20 sec; and plate 16, 30 sec.

The UV treatment was based on the reference from Levin et. al., PNAS 79, 7445-9,1982. A germicidal UV source was used (Alpha Innotech Corporation, San Leandro, Calif., Image Analyzer, UV source of 15W) at an approximate distance of 33 cm from the glass petri dish (3 Joules/meters exp2/sec).

UV irradiation is considered to lead to mutations due to the formation of thymine dimers. The results indicated a correlation between traditional Ames His-plates and the level of heteroduplex as determined using DHPLC. An increase in revertants as indicated by the Ames modified plate assay at longer periods of UV radiation was observed, which paralleled an increase in the level of heteroduplex detected.

### EXAMPLE 3

Evaluation of Mutagenicity of Daunomycin in Strain TA98

TA98 were prepared in a 100 ul first stage incubation as described in example 1 in the presence of different amounts of daunomycin form 0 to 0.3 ug. A second stage incubation was also performed as in example 1, followed by PCR and sequencing analysis for mutation detection.

For TA98, the following 397 bp amplicon from the his D gene was prepared:

(SEQ ID NO.6)

GTCTGAAGTACTGGTGATCGCAGACAGCGGCGCAACACCGGATTTCGTCG
CTTCTGAGCTGCTCTCCCAGGGTGAGCACGGCCCGGATTCCCAGGTGATC
CTGCTGACGGGTGATGCTGACATTGCCCGCAAGGTGGCGGAGGCGGTAGA
ACGTCAAGTGGCGGAACTGCCGCGCGCGGACACCGCCGGGAGGCGCTGAG
CGGGAGTCGTCTGATTGTGACCAAAGATTTAGCGCAGTGGGTCGCCATCT
CTAATCAGTATGGGCCGGAACACTTAATCATCCAGACGGGCAATGCGCGC
GATTTGGTGGATGCGATTACCAGCGCAGGGTCGGTATTTCTCGGCGACTG
GTCGCCGGAATCCGCCGGTGATTACGCTTCCGGAACCAACCATGTT

(The original mutation used to create the tester strain is underlined; it represents the deletion of a C residue from CCC to CC which is underlined. A major mutation hot spot is represented by a 4CG repeat (shown in italics), which changes to a 3CG in revertants).

In the PCR, the forward primer was GTCTGAAGTACTGGTGATCGCAGA (SEQ ID NO: 7), and the reverse primer was AACATGGTTGGTTCCGGAAGCGTA (SEQ ID NO: 8). PCR conditions were as follows:

| Stage | Temp. | | Duration |
|---|---|---|---|
| 1. | 95° C. | | 20:00 |
| 2. | 95° C. | | 00:30 |
| 3. | 67° C. | | 01:00 |
| | −0.5° C. | | |
| | per cycle | | |
| 4. | 72° C. | | 01:00 |
| 5. | go to 2 | 15 | |
| | | times | |
| 6. | 95° C. | | 00:30 |
| 7. | 62° C. | | 01:00 |
| 8. | 72° C. | | 01:00 |
| 9. | go to 6 | 20 | |
| | | times | |
| 10. | 72° C. | | 05:00 |
| 11. | 4° C. | | storage |

Sequence analysis of the PCR product was performed by Retrogen Inc. (San Diego, Calif.) using the forward primer after it was cleaned with the SeqDirect PCR Cleaning kit from Qbiogene (Carlsbad, Calif.). Data indicated a CG deletion when cells were exposed to the mutagen. The zero mutagen control (vehicle control) did not show the deletion (data not shown).

### EXAMPLE 4

Petroleum Extract Analysis

In the following example, petroleum extracts are evaluated for mutagenicity. The following materials (Sterile solutions) are prepared:

TA98 culture (18 h growth, LB/Ampicillin 20 ug/ml) MOLTOX (Boone, N.C.);

1M sodium phosphate solution, pH 7.4 (Sigma);

1M Glucose-6-phosphate (MOLTOX);

0.2M NADP (MOLTOX);

1.65M KCl (Sigma)/0.4M MgCl₂ (Sigma);

HC235 extract (kindly provided by Gary Blackburn, Petron, Columbus, Ohio) (control);

DMSO (Sigma);

Ampicillin, 8 mg/ml (MOLTOX);

M9 minimal salts Media (Parts A and B) (Qbiogene, Carlsbad, Calif., Lot No. 3035-012-87617);

D-+-Glucose (Sigma, Catalog no. G-7258, Lot no. 101KO149);

Nutrient Agar—Oxoid No.2 25 g/l (Lot no. 25815, MOLTOX);

CSM-His (Catalog no.4510-312, Qbiogene);

d-Biotin (Vitamin H) (Sigma, Catalog no. B-4501, Lot no. 121K1505);

L-Histidine (Sigma, Catalog no. H-6034, Lot no. 31K0891);

(Traditional Blackburn Modified Ames: HB Top Agar (Catalog no. 26-503.3, MOLTOX)).

1. To 8 ml of nutrient agar broth, is added 26 ul of Ampicillin (8 mg/ml, 20 ug/ml final concentration) and 2 ml of TA98 18 h culture.

2. The mixture is incubated for 3 h, 35° C., 150 rpm until turbidity is 1.0 to 1.4 absorbance units at 650 nm.

3. Seven rows of sterile Eppendorf tubes, four tubes/row, are set up.

4. Seven dosing solutions are prepared, vortexing to mix:

| | | | | | | | |
|---|---|---|---|---|---|---|---|
| DOSE (final | 0 | 5 | 10 | 15 | 20 | 30 | 40 |
| ul oil | | | | | | | |
| extract/sample) | | | | | | | |
| Oil Extract | 0 | 20 | 40 | 60 | 80 | 120 | 160 |
| (ul) | | | | | | | |
| DMSO (ul) | 240 | 220 | 200 | 180 | 160 | 120 | 80 |

5. 60 ul of each dosing solution is transferred into the three corresponding tubes for each dose.

6. The following core/S9 mixture is prepared on ice in a sterile tube. Per S9 to assayed, the following are added in order (to be used within one hour of preparation):

| Final Vol. | Final Volume | | (/10) | |
|---|---|---|---|---|
| 1 M phosphate buffer | 1.5 | ml | 0.15 | ml |
| 1 M glucose-6- | 0.075 | ml | 0.075 | ml |
| phosphate | | | | |
| water | 0.527 | ml | 0.053 | ml |
| 0.2 M NADP | 0.598 | ml | 0.06 | ml |
| 0.4 M MgCl₂/1.65 M KCl | 0.300 | ml | 0.03 | ml |
| S9 | 12 | ml | 1.2 | ml |
| | 15 | ml | 1.5 | ml final |
| | (use 0.5 | ml/sample) | (0.02 | ml/sample) |

7. 0.5 ml S9/core mixture is added to each of the dosed tubes (21 total).

8. 0.1 ml of re-grown TA98 is added to each tube.

9. Each tube is briefly vortexed and incubated at 35° C., 150 rpm for 20 min.

10. Minimal glucose plates are labeled (21 total).

11. 2 ml melted/cooled (35° C.) top agar is added to each sample, vortexed and poured evenly over appropriate plate. When plates have set, they are taped together, inverted, and incubated (35° C., 48 h).

| Final | | | | | |
|---|---|---|---|---|---|
| sample: | Blackburn | | | Modification | |
| | 0.06 ml | | /6 | 0.01 mL dosin | |
| | dosing solution | | | solution | |
| | 0.5 ml S9/ | | /30 | 0.015 mL | |
| | core mixture | | | S9/water | |
| | 0.1 ml TA98 | 0.66 ml | /100 | 0.001 mL TA98 | 0.026 mL |
| | 2 ml top agar | | | | |
| | Add to | | | | |
| | His- plate | | | | |

The following is a modification of Blackburn Oil Extract Analysis. The reactions are performed in sterile 1.5 ml Eppendorf tubes:

| | | |
|---|---|---|
| TA98 (18 h culture) | 1 | ul |
| LB nutrient broth | 7 | ul |
| S9 mix/water | 20 | ul (40 ul, 60 ul) |
| M9-Histidine + Biotin | 62 | ul (42 ul, 22 ul) |
| Test compound | 10 | ul |
| | 100 | ul sample |

Each tube is vortexed briefly and incubated at 1.5 h, 37° C. 800 ul M9-Histidine plus Biotin is added and the mixture incubated overnight, 37° C. PCR is performed using the protocol indicated in Example 1 and heteroduplex analysis is performed by DHPLC.

### EXAMPLE 5

Thirteen petroleum hydrocarbon oil samples and a corn oil sample, are assayed for the formation of heteroduplex by DHPLC using strains TA102 and TA98 by the method described in Example 4. The samples are described in Table 3.

TABLE 3

| | | Chemical |
|---|---|---|
| Example | Sample Tested | Abstract No. |
| 6 | Mix of Heavy Catalytic Cracked | 64741-61-3 |
| 7 | Distillate & Catalytic Cracked | 64741-62-4 |
| 8 | Intermediate Catalytic Cracked | 64741-60-2 |
| | Distillate | |
| 9 | Light Paraffinic Distillate | 64741-50-0 |
| 10 | Hydrotreated Heavy Naphthenic | 64742-52-5 |
| | Distillate | |
| 11 | Chemically | 64742-34-3/ |
| | Neutralized/Hydrotreated (100″) Heavy | 64742-52-5 |
| | Naphthenic Distillate | |
| 12 | Hydrotreated Heavy Naphthenic | 64742-52-5 |
| | Distillate | |
| 13 | Solvent Refined/Hydrotreated | 64741-96-4/ |
| | Heavy Naphthenic Distillate | 64742-52-5 |
| 14 | Solvent Refined/Dewaxed | 64742-01-4/ |
| | Residual Oil | 64742-62-7 |
| 15 | Solvent Refined/Hydrotreated | |
| | (430″) Heavy Paraffinic Distillate | |
| 16 | Solvent Refined/Hydrotreated | 64741-88-4/ |
| | (400″) Heavy Paraffinic Distillate | 64742-54-7 |
| 17 | Solvent Refined/Hydrotreated (300″) | |
| | Heavy Paraffinic Distillate | |
| 18 | Solvent Refined/Hydrotreated | 64742-01-4/ |
| | Residual Oil | 64742-57-0 |
| 19 | Solvent Refined/Dewaxed | 64741-88-4/ |
| | Heavy Paraffinic Distillate | 64742-65-0 |
| 20 | Corn Oil | |

While the foregoing has presented specific embodiments of the present invention, it is to be understood that these embodiments have been presented by way of example only. It is expected that others will perceive and practice variations which, though differing from the foregoing, do not depart from the spirit and scope of the invention as described and claimed herein.

All patent applications, patents, and literature references cited in this specification are hereby incorporated by reference in their entirety. In case of conflict or inconsistency, the present description, including definitions, will control.

The invention claimed is:

1. A method for screening a test substance suspected of being a mutagen, the method comprising:
a) providing a tester strain of Salmonella typhimurium,
wherein said tester strain comprises a histidine gene having a preexisting mutation conferring auxotrophy, said mutation located at a pre-determined position in said gene,
b) determining a first level of back-mutation in the nucleic acid sequence of said gene in tester strain which have not been exposed to said substance,
c) exposing tester strain to said substance and determining a second level of back-mutation in the nucleic acid sequence of said gene,
d) comparing said first level with said second level.

2. The method of claim 1 wherein said preexisting mutation is a base substitution or a frame shift mutation.

3. The method of claim 1 including amplifying a pre-selected region of said histidine gene, wherein said position is within said region.

4. The method of claim 3 wherein said amplifying is by the polymerase chain reaction.

5. The method of claim 1 wherein determining in step (b) and step (c) includes a method selected from the group consisting of direct sequencing, minisequencing, pyrosequencing, single-stranded conformation polymorphism, denaturing gradient gel electrophoresis, chemical cleavage, cleavage by mismatch endonuclease, allele specific oligonucleotides, ligase mediated detection of mutations, invader assay, and denaturing high performance liquid chromatography.

6. The method of claim 1 wherein determining in step (b) and step (c) includes performing denaturing high performance liquid chromatography.

7. The method of claim 1 wherein said test substance is selected from the group consisting of petroleum extract, pesticides, cosmetics, adhesives, food coloring, herbicides, hair dyes, and pharmaceuticals.

8. The method of claim 1 wherein said test substance comprises petroleum extract.

9. The method of claim 1 wherein said tester strain is selected from the group consisting of TA98, TA100, TA102, TA104, TA1535, TA1537, TA1538, and TA97.

10. The method of claim 1 wherein determining in step (b) and step (c) includes S9 homogenate.

11. The method of claim 1 further including correlating the level of DNA having said back-mutation with the concentration of said substance in said media.

12. The method of claim 1 wherein step (d) further includes determining whether said test substance is a mutagen.

13. The method of claim 1 wherein said test compound comprises a hydrocarbon mixture, and wherein said method includes extracting the hydrocarbon mixture with a solvent effective for removing mutagenic compounds from said mixture.

14. The method of claim 13 wherein an optimal amount of induced liver homogenate is included as a metabolic activator in said incubation.

15. The method described in claim 13 wherein said solvent is DMSO, said mutant strain is Salmonella typhimurium TA98, and said liver homogenate is Aroclor 1254-induced rat S9.

16. The method described in claim 13 wherein said solvent is DMSO, said mutant strain is Salmonella typhimurium TA98, and said liver homogenate is Aroclor 1254-induced hamster S9.

17. The method described in claim 13 wherein said solvent is DMSO, said mutant strain is Salmonella typhimurium TA102, and said liver homogenate is Aroclor 1254-induced rat S9.

18. The method described in claim 13 wherein said solvent is DMSO, said mutant strain is Salmonella typhimurium TA102, and said liver homogenate is Aroclor 1254-induced hamster S9.

19. The method described in claim 13 wherein said hydrocarbon mixture is of petroleum origin.

20. The method described in claim 13 wherein said solvent is selected from the group consisting of DMSO, 1-methyl-2-pyrrolidinone and N,N-dimethylformamide, said mutant strain is selected from the group consisting of Salmonella typhimurium TA98 and TA102, and said induced liver homogenate is selected from the group consisting of Aroclor 1254-induced rat liver S9 and Aroclor 1254-induced hamster liver S9.

21. A method for determining the mutagenic potential of a putative mutagen, the method comprising:
a) exposing a tester strain of Salmonella typhimurium to said mutagen,
wherein said tester strain comprises a histidine gene having a preexisting mutation conferring auxotrophy, said mutation located at a predetermined position in said gene,
b) growing said tester strain in growth media lacking histidine,
c) detecting the presence of a back-mutation at said position, wherein the presence of said back-mutation is correlated with the mutagenic potential of said mutagen.

22. The method of claim 21 wherein said putative mutagen comprises ultraviolet light.

23. A method for evaluating the potential mutagenicity of a test compound, which method comprises:
a) subjecting an inoculum of a histidine deficient mutant strain of Salmonella typhimurium to incubation in the presence of said compound wherein said strain comprises DNA possessing a preexisting mutation at a pre-determined position in the histidine biosynthetic operon,
b) determining the presence in said incubation of DNA having a back-mutation at said position, wherein said DNA having a back-mutation is indicative of the mutagenicity of said compound.

24. The method of claim 23 wherein said test compound comprises a hydrocarbon mixture, and wherein said method includes extracting the hydrocarbon mixture with a solvent effective for removing mutagenic compounds from said mixture.

25. The method of claim 23 wherein an optimal amount of induced liver homogenate is included as a metabolic activator in said incubation.

26. The method described in claim 24 wherein said hydrocarbon mixture is of petroleum origin.

27. A method for evaluating the potential mutagenicity of a hydrocarbon mixture, which method comprises:
extracting the hydrocarbon mixture with a solvent effective for removing mutagenic compounds from said mixture, wherein an extract is obtained;
subjecting an inoculum of a histidine deficient mutant strain of Salmonella typhimurium to incubation in the presence of a sample of said extract and, as metabolic activator, an optimal amount of induced liver homogenate, wherein said strain comprises DNA possessing a preexisting mutation at a pre-determined position in the histidine biosynthetic operon;
determining the presence in said incubation of DNA having a back-mutation at said position, wherein said DNA having a back-mutation is indicative of the mutagenicity of said mixture.

28. A method for screening a hydrocarbon mixture suspected of being a mutagen, the method comprising:
a) extracting the hydrocarbon mixture with a solvent effective for removing mutagenic compounds from said mixture, wherein an extract is obtained;
b) providing a tester strain of Salmonella typhimurium,
wherein said tester strain comprises a histidine gene having a preexisting mutation conferring auxotrophy, said mutation located at a pre-determined position in said gene,
c) determining a first level of back-mutation in the nucleic acid sequence of said gene in tester strain which have not been exposed to said extract,
d) exposing tester strain to said extract and determining a second level of back-mutation in the nucleic acid sequence of said gene,
e) comparing said first level with said second level.

29. A method for determining the mutagenic potential of a petroleum extract, the method comprising:
a) exposing a tester strain of Salmonella typhimurium to said extract,
wherein said tester strain comprises a histidine gene having a preexisting mutation conferring auxotrophy, said mutation located at a predetermined position in the nucleic acid sequence of said gene,
b) growing said tester strain in growth media lacking histidine,
c) detecting the presence of a back-mutation in the nucleic acid sequence at said position, wherein said extract is classified as a mutagen if the level of back-mutation is above a pre-determined background level.

30. A method for evaluating a test substance suspected of being a mutagen, the method comprising:
a) exposing a tester strain of Salmonella typhimurium to said substance,
wherein said tester strain comprises a histidine gene having a preexisting mutation conferring auxotrophy, said mutation located at a pre-determined position in said gene,
b) growing said tester strain in growth media lacking histidine, and
c) detecting the presence of a back-mutation at said position, wherein said substance is classified as a mutagen if the level of said back-mutation is above a pre-determined background level.

31. A method for determining the mutagenic potential of a test substance, the method comprising:
a) a step for exposing a tester strain of Salmonella typhimurium to said substance,
wherein said tester strain comprises a histidine gene having a preexisting mutation conferring auxotrophy, said mutation located at a pre-determined position in said gene,
b) a step for growing said tester strain in growth media lacking histidine, and
c) a step for detecting the presence of a back-mutation at said position, wherein the presence of said back-mutation correlates with the mutagenic potential of said substance.

32. A method for screening a test substance suspected of being a mutagen, the method comprising:
a) a step for providing a tester strain of Salmonella typhimurium,
wherein said tester strain comprises a histidine gene having a preexisting mutation conferring auxotrophy, said mutation located at a pre-determined position in said gene,
b) a step for determining a first level of back-mutation in the nucleic acid sequence of said gene in tester strain which have not been exposed to said substance,
c) a step for exposing tester strain to said substance and determining a second level of back-mutation in the nucleic acid sequence of said gene,
d) a step for comparing said first level with said second level.

33. A kit for evaluating the mutagenicity of a test compound, said kit comprising, in separate containers:
a tester strain of Salmonella typhimurium, wherein said tester strain is selected from the group consisting of TA98, TA100, TA102, TA104, TA1535, TA1537, TA1538, and TA97, and
pre-selected PCR primers specific for amplification of a region of the Salmonella histidine operon in said strain, wherein said region contains a preexisting mutation that confers auxotrophy in said strain, wherein said primers generate an amplicon having a length of about 150 base pairs to about 500 base pairs.

34. The kit of claim 33 further including a reference DNA fragment corresponding to said region for use in a hybridization protocol with said amplicon.

35. The kit of claim 33 further including S9 fraction in a separate container.

36. The kit of claim 33 further including a positive control compound.

37. The kit of claim 36 wherein said control compound includes HC235.

38. The kit of claim 33 further including a proofreading DNA polymerase.

39. The kit of claim 38 wherein said polymerase comprises Pho polymerase.

40. The kit of claim 38 wherein said polymerase comprises Taq polymerase.

41. A kit for evaluating the mutagenicity of a test compound, said kit comprising:
pre-selected PCR primers specific for amplification of a region of the Salmonella histidine operon in said strain, wherein said region contains a preexisting mutation that confers auxotrophy in said strain, wherein said primers generate an amplicon having a length of about 150 base pairs to about 500 base pairs.

42. The kit of claim 41 further including a reference DNA fragment corresponding to said region for use in a hybridization protocol in DHPLC.

43. A method for determining the mutagenic potential of a test substance, the method comprising:
a) exposing a bacterial tester strain to said substance in a first media,
wherein said tester strain comprises a gene having a preexisting mutation conferring auxotrophy, said mutation located at a pre-determined position in said gene,
b) growing said tester strain in selective enrichment media to enrich for back-mutation revertants, and
c) detecting the presence of a back-mutation, at the nucleic acid level at said position or nearby, wherein the presence of said back-mutation is distinguished from spontaneous revertant mutations and correlates with the mutagenic potential of said substance.

44. The method of claim 43 wherein said tester strain comprises Salmonella and said gene comprises the histidine biosynthetic gene.

45. The method of claim 43 wherein said tester bacteria comprises E. coli and said gene comprises the tryptophan biosynthetic gene.

46. The method of claim 43 wherein step (a) and step (b) are combined into a single step.

47. The method of claim 43 wherein said first media and said selective enrichment media are devoid of histidine.

48. The method of claim 43 wherein said first media and said selective enrichment media contain a limiting amount of histidine.

49. The method of claim 43 wherein said first media and said selective enrichment media contain a limiting amount of Luria Broth.

50. The method of claim 43 wherein said first media and said selective enrichment media contain a limiting amount of tester strain growth media to support limited growth of said tester strain while allowing selective enrichment of said back-mutation revertants.

51. The method of claim 43 wherein enrichment of said back-mutation revertants occurs to a level sufficient for detection of said back mutation at the nucleic acid level.

52. The method of claim 51 wherein said detection includes a method selected from the group consisting of direct sequencing, minisequencing, pyrosequencing, single-stranded conformation polymorphism, denaturing gradient gel electrophoresis, chemical cleavage, cleavage by mismatch recognition endonuclease, allele specific oligonucleotides, ligase mediated detection of mutations, invader assay, sizing, and denaturing high performance liquid chromatography.

53. The method of claim 43 wherein a plurality of tester strains are used in step (a).

54. The method of claim 43 wherein said method is repeated at different doses of said test substance.

55. The method of claim 43 wherein said tester strain is used in limited amounts to control the level of said background revertants.

56. The method of claim 43 wherein said tester strain is exposed multiple independent times to said test substance to account for said background revertants.

57. The method of claim 56 wherein said multiple independent times represent a statistically significant number of repeats to establish a 2-fold increase over said background revertants.

58. The method of claim 43 wherein said tester strain comprises a plurality of tester strains, in any combination, selected from two or more of the group consisting of TA98, TA100, TA102, TA1535, TA1537, TA1538, and TA97.

59. The method of claim 58 wherein said tester strain comprises a limited amount of said plurality of tester strains to control the level of said background revertants.

60. The method of claim 43 wherein said back-mutation comprises an insertion or deletion.

61. The method of claim 43 wherein said test substance is dissolved in liquid solvent selected the group consisting of DMSO, glycerol formal, dimethyl formamide, formamide, acetonitrile, 95% ethanol, acetone, ethyl glycol, dimethyl ether, 1-methyl-2-pyrrolidone, tetrahydrofurfuryl alcohol, water, and tetrahydrofuran.

62. The method of claim 61 wherein said liquid solvent is used at a concentration that is not lethal to tester strain.

63. A kit for evaluating the mutagenicity of a test compound, said kit comprising in a separate container:
a plurality of tester strains of Salmonella typhimurium, wherein said tester strains are selected from two or more of the group consisting of TA98, TA100, TA102, TA104, TA1535, TA1537, TA1538, and TA97.

64. The kit of claim 63 where the said tester strains are provided in a ready-to-use format.
